(19) United States
(12) Patent Application Publication
(10) Pub. No.: US 2022/0362752 A1
(43) Pub. Date: Nov. 17, 2022

(54) PROCESSES FOR CATALYTICALLY COATING SCAFFOLDS

(71) Applicant: COMMONWEALTH SCIENTIFIC AND INDUSTRIAL RESEARCH ORGANISATION, Acton, Australian Capital Territory (AU)

(72) Inventors: Zongli Xie, Clayton, Victoria (AU); Christian Harald Hornung, Clayton, Victoria (AU); Choon Chian Ng, Clayton, Victoria (AU); Xingdong Wang, Clayton, Victoria (AU)

(21) Appl. No.: 17/621,117

(22) PCT Filed: May 11, 2020
(86) PCT No.: PCT/AU2020/050464

§ 371 Date: Dec. 20, 2021

(30) Foreign Application Priority Data
Jun. 21, 2019 (AU) 2019902166

(51) Int. Cl. B01J 29/06; B01J 21/18; B01J 23/46; B01J 23/44; B01J 23/755; B01J 23/14; B01J 21/04; B01J 21/06; B01J 21/12; B01J 27/232; B01J 35/02; B01J 37/02; B01J 37/00; B01J 37/08; B01J 10/00; B01J 19/24; C07C 29/17

(52) U.S. Cl. CPC B01J 29/06; B01J 21/18; B01J 23/462; B01J 23/44; B01J 23/755; B01J 23/14; B01J 21/04; B01J 21/06; B01J 21/12; B01J 27/232; B01J 35/026; B01J 37/0225; B01J 37/0217; B01J 37/0219; B01J 37/0236; B01J 37/0018; B01J 37/08; B01J 10/007; B01J 19/248; C07C 29/17

(57) ABSTRACT

The present disclosure generally relates to a process for coating a scaffold, and in particular a process for coating a scaffold of a static mixer using catalytic liquid suspensions. The present disclosure also generally relates to a process for preparing a catalytically coated scaffold comprising applying a catalytic liquid suspension to a surface of a scaffold to provide a coating containing catalytically reactive sites on the surface of the coated scaffold.

### FIELD

The present disclosure generally relates to a process for coating a scaffold, and in particular a process for coating a scaffold of a static mixer using catalytic liquid suspensions.

### BACKGROUND

Continuous flow chemical reactors generally comprise a tubular, ducted, plate-type channel or chip-type channel reaction chamber with reactant fluids being continuously fed into the reaction chamber to undergo a chemical reaction to continuously form products which flow out from the reaction chamber. The reaction chambers are typically heated electrically or by means of a recirculating heating/coolant fluid, for example in a shell-and-tube heat exchanger configuration, to facilitate the transfer of heat to/away from the reaction.

Continuous flow reactors used in catalytic reactions typically employ packed bed reaction chambers in which the reaction chamber is packed with solid catalyst particles that provide catalytic surfaces on which the chemical reaction can occur. Static mixers are used for pre-mixing of fluid streams prior to contact with the packed bed reaction chambers and downstream of these chambers to transfer heat between the central and the outer regions of the reactor tubes. The static mixers comprise solid structures that interrupt the fluid flow to promote mixing of the reactants prior to reaction in the packed bed reaction chambers and for promoting desirable patterns of heat transfer downstream of these chambers.

There is a need for alternative or improved processes for catalytically coating scaffolds, and in particular scaffolds of static mixers, that can provide various desirable properties such as flexibility and usability of catalytic static mixer technology which are capable of providing more efficient mixing, heat transfer and catalytic reaction of reactant chemical and/or electrochemical reactants.

### SUMMARY

The present inventors have undertaken significant research and development into alternative catalyst deposition methods and have identified that the surface of static mixer scaffolds can be provided with a catalytic surface such that the resulting static mixer is capable of being used with a continuous flow chemical reactor.

In one aspect, there is provided a process for preparing a catalytically coated scaffold, comprising the step of: (i) applying a catalytic liquid suspension to a surface of a scaffold to provide a coating containing catalytically reactive sites on the surface of the coated scaffold, wherein the catalytic liquid suspension comprises a liquid carrier containing a plurality of ex-situ catalyst particles, and wherein the coated scaffold has a non-line-of-sight configuration comprising a plurality of passages configured for dispersing and mixing one or more fluidic reactants during flow and reaction thereof, and (ii) drying the coated scaffold to remove the liquid carrier to provide a coated scaffold comprising ex-situ catalyst particles. In an embodiment, the scaffold may be a static mixer. In an embodiment, the surface of the static mixer may be pre-coated before step (i) with a support material and optionally a binder. In an example, the catalytic liquid suspension further comprises a binder. In an embodiment, the step of applying the catalytic liquid suspension to the surface of the scaffold in step (i) may be by wash-coating or dip-coating.

In another embodiment, the process includes a pre-treatment step prior to applying the catalytic liquid suspension to the surface of the scaffold in step (i), wherein the pre-treatment step may be at least one surface treatment step to the surface of the scaffold selected from chemical treatment, anodic oxidation, hot dipping, vacuum plating, painting, thermal spraying, and acid etching.

In an embodiment, the catalyst particles are formed from a catalyst material or a catalyst supported material comprising the catalyst material on a support material. The catalyst material may be selected from a metal, metal oxide, aluminium silicate, activated carbon, mesoporous carbon, graphene, graphitic material, metal-organic framework, zeolite, or any combination thereof. For example, the metal may be selected from at least one of aluminium, iron, cerium, calcium, cobalt, copper, magnesium, zinc, nickel, palladium, platinum, gold, silicon, silver, ruthenium, iridium, rhodium, titanium, vanadium, zirconium, niobium, tantalum, and chromium, or a metal oxide thereof.

The catalyst supported material may be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite.

The support material may be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide. When a catalyst material is used as a support material, the catalyst material and support material would be different.

In an embodiment, the concentration of the catalyst particles in the catalytic liquid suspension may be less than 10 wt. % based on the total weight of the catalytic liquid suspension.

In another embodiment, the binder for the catalytic liquid suspension may be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide, or polyisobutylene. The binder may be added at a concentration of between about 0.3 wt. % to about 5 wt. % based on the total weight of the catalytic liquid suspension.

In an embodiment, the liquid carrier may be selected from the group comprising water, ethanol, isopropanol, butanol, ethyl acetate, acetone or a combination thereof.

In an embodiment, the catalytic liquid suspension may have a solids content of between about 3 wt. % to about 28 wt. %. The thickness of the coating may be between about 1 μm to about 50 μm. The surface area of the catalyst may be between about 1 m²/g to about 1000 m²/g. The adhesion of the coating may provide a total mass loss of the coating of less than about 0.5 wt. % when measured by sonication testing.

In an embodiment, the scaffold may be a metal, metal alloy, cermet, carbon fibre, silicon carbide or polymer. In an example, the scaffold may be a metal scaffold. For example, the metal or metal alloy of the metal scaffold is titanium, aluminium or stainless steel.

In an embodiment, the aspect ratio (L/d) of the scaffold may be at least 75.

In another embodiment, the ex-situ catalyst particles may be less than about 5 μm.

In an embodiment, the process may further comprise a drying step and/or an activation step. The drying step may comprise the steps of: (a) applying a first temperature ranging between about 15° C. to about 30° C. to the coated surface of the scaffold for a first period of about 4 to 24 hours to volatilise at least a portion of volatile material from the catalytic liquid suspension; and (b) applying a second temperature ranging between about 100° C. to about 180° C. under controlled atmosphere for a second period of about 4 to 24 hours such that a dried coating is formed on the surface of the scaffold.

In another aspect there is provided a catalytically coated scaffold prepared by the process for preparing the catalytically coated scaffold as defined herein.

In another aspect there is provided a catalytically coated scaffold comprising a coating on a scaffold, wherein the coating comprises a plurality of catalyst particles, and wherein the coated scaffold has a non-line-of-sight configuration comprising a plurality of passages configured for dispersing and mixing one or more fluidic reactants during flow and reaction thereof. In an embodiment, the coating comprises a support material and optionally a binder.

In an embodiment, the catalyst particles may be selected from a metal, metal oxide, aluminium silicate, activated carbon, mesoporous carbon, graphene, graphitic material, metal-organic framework, zeolite, or any combination thereof. The catalyst particles may be a metal selected from at least one of aluminium, iron, cerium, calcium, cobalt, copper, magnesium, zinc, nickel, palladium, platinum, gold, silicon, silver, ruthenium, iridium, rhodium, titanium, vanadium, zirconium, niobium, tantalum, and chromium, or a metal oxide thereof.

In an embodiment, the support material may be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide.

In an embodiment, the catalyst supported material may be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite.

In an embodiment, the binder may be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide, or polyisobutylene.

In an embodiment, the catalyst particles may be less than 5 μm. The thickness of the coating may be between about 1 μm to about 50 μm.

In an embodiment, the coated scaffold may be a coated static mixer scaffold.

In another aspect there is provided, a continuous flow chemical reactor for use in reaction of one or more fluidic reactants, the reactor comprising one or more catalytically coated scaffolds prepared by the process as defined herein or the catalytically coated scaffold as defined herein. The one or more fluidic reactants may be provided as a continuous fluidic stream. The continuous fluidic stream may be provided by at least one liquid phase.

In another aspect there is provided a continuous flow process for a heterogeneous reaction comprising one or more chemical reactors as defined herein.

### BRIEF DESCRIPTION OF THE DRAWINGS

Preferred embodiments of the present disclosure will now be further described and illustrated, by way of example only, with reference to the accompanying drawings in which:

FIG. 1 shows routes for coating a static mixer scaffold via options (a) to (d).

### DETAILED DESCRIPTION

The present disclosure describes the following various non-limiting embodiments, which relate to investigations undertaken to identify alternative or improved processes for coating scaffolds of static mixers that can provide various desirable properties such as flexibility and usability of catalytic static mixer technology which are capable of providing more efficient mixing, heat transfer and catalytic reaction of reactant chemical and/or electrochemical reactants. It was surprisingly found that depositing catalytic material on the surface of additive manufactured static mixers can provide efficient mixing, heat transfer and catalytic reaction of reactants in continuous flow chemical reactors. It will be appreciated that the catalyst deposition technique described by the present invention may depend on the application and the type of catalyst employed. The inventors have surprisingly identified that the catalyst material, as described herein, provides an improved catalytic deposition technique for coating complex three-dimensional structures, such as static mixer scaffolds.

Compared to current heterogeneous catalysis systems, such as packed beds, the present static mixers have been shown to provide various advantages. Additive manufacturing technology (i.e. 3D printing) enables flexibility in re-design and configuration of the static mixers, although presents other difficulties and challenges in providing robust commercially viable scaffolds that can be catalytically coated to operate under certain operational performance parameters of continuous flow chemical reactors, such as to provide desirable mixing and flow conditions inside the continuous flow reactor, and enhanced heat and mass transfer characteristics and reduced back pressures compared to packed bed systems.

Wash-coating and dip-coating techniques have been found to be surprisingly suitable for catalytically coating the static mixer scaffolds and suitable for application with a wide variety of catalyst materials.

The static mixers can be configured as scaffolds to provide inserts for use with in-line continuous flow reactor systems. The static mixers can also provide heterogeneous catalysis, which is of significant importance to chemical manufacturing and is broad ranging including the production of fine and specialty chemicals, pharmaceuticals, food and agrochemicals, consumer products, and petrochemicals.

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Those skilled in the art will appreciate that the disclosure herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated scaffold, integer or step, or group of scaffolds, integers or steps, but not the exclusion of any other scaffold, integer or step, or group of scaffolds, integers or steps. It will be clearly understood that, although a number of prior art publications are referred to herein, this reference does not constitute an admission that any of these documents forms part of the common general knowledge in the art, in Australia or in any other country.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

### Coating Process

In one embodiment or example, there is provided a process for preparing a catalytically coated scaffold, comprising the step of: (i) applying a catalytic liquid suspension to a surface of a scaffold to provide a coating containing catalytically reactive sites on the surface of the scaffold, wherein the catalytic liquid suspension comprises a liquid carrier containing a plurality of catalyst particles of less than about 5 μm. In an embodiment, the scaffold may be a static mixer. In an embodiment or example, the surface of the static mixer may be pre-coated before step (i) with a support material and optionally a binder. In an example, the catalytic liquid suspension further comprises a binder. In an embodiment, the step of applying the catalytic liquid suspension to the surface of the scaffold in step (i) may be by wash-coating or dip-coating.

In another embodiment or example, a process for coating a scaffold of a static mixer, may comprise the step of: (i) applying a catalytic liquid suspension to the surface of the scaffold for providing the surface of the scaffold with catalytically reactive sites, wherein the catalytic liquid suspension comprises a catalyst particles with particle sizes of less than about 5 μm and a liquid carrier, and wherein the surface of the static mixer is (a) a static mixer scaffold, (b) a static mixer scaffold comprising a support material, or (c) a static mixer scaffold comprising a support material and a binder. The catalytic liquid suspension may further comprises a binder.

In an embodiment or example, the step of applying the catalytic liquid suspension to the surface of the scaffold in step (i) may be by wash-coating or dip-coating. The inventors have surprisingly shown that the wash-coating or dip-coating deposition technique provides advantages for the catalytic static mixers such as, for example, an effective and scalable method for the deposition of the catalyst material onto static mixer scaffold by wash-coating/dip-coating the static mixer in a catalytic liquid suspension. This technique may include incorporation of binding material to facilitate better adhesion or improve specific functionality via addition of Teflon to increase hydrophobicity, with the advantage of smaller diffusional distance towards active phase. It will be appreciated that standard catalyst coating techniques have typically involved a two-step process of first forming a porous metal oxide layer and then secondly impregnating the catalyst into the pre-formed porous layer. Other coating techniques such as electrodeposition and cold spray techniques are limited by the type of metal that can be coated and the size of the metal to be coated. Other limitations include line-of-sight configurations which impose constraints on the coating technique. For example, line-of-sight coating techniques generally allow coverage of exposed areas of a surface of a scaffold, i.e. twisted plate configuration. Several methods have been reported to deposit coatings onto various scaffolds. Cold spray techniques are frequently used but only the exposed surfaces of the scaffold can be coated, therefore, coverage of the coating is incomplete and inhomogeneous, with some areas remaining uncovered

It will be appreciated that the inventors have shown that wash-coating or dip-coating deposition techniques, as described herein, provide for more complete coverage of even complex scaffold configurations. In other words, the wash-coating or dip-coating deposition technique is a non-line-of-sight coating technique which can provide for more complete coverage of the surface of the scaffold. For example, a scaffold comprising a complex geometry with shaded areas can be referred to as a non-line-of-sight configuration or non-line-of-sight scaffold.

In an embodiment or example, the process may include a pre-treatment step prior to applying the liquid suspension to the surface of the scaffold in step (i), wherein the pre-treatment step may be at least one surface treatment step selected from chemical treatment, anodic oxidation, hot dipping, vacuum plating, painting, thermal spraying, and acid etching.

In an example, chemical treatment may involve processes that create thin films of sulphide and oxide by means of a chemical reaction. Typical uses are for metal colouring, corrosion protection, and priming of surfaces to be painted. Black oxide is a very common surface treatment for steel parts and “passivation” is used to remove free iron from the surface of stainless steel parts.

In another example, anodic oxidation may typically be used for light metals such as aluminium and titanium. These oxide films are formed by electrolysis, and since they are porous, dyeing and colouring agents are frequently specified for an improved aesthetic appearance. Anodization is a very common surface treatment that prevents corrosion on aluminium parts. If wear resistance is also desirable, engineers can specify a version of this method that forms a relatively thick, extremely hard, ceramic coating on the surface of the part.

In another example, hot dipping may require the scaffold to be dipped into dissolved tin, lead, zinc, aluminium, or solder to form a surface metallic film. Hot-dip galvanizing is the process of dipping steel into a vessel containing molten zinc and typically used for corrosion resistance in extreme environments, guard rails on roads are commonly processed with this surface treatment.

In another example, vacuum plating may be used. Vacuum vapour deposition, sputtering, ion plating, ion nitriding, and ion implantation are the typical metal surface finishing processes utilizing high vacuum as part of the plating process. Ionized metals, oxides, and nitrides are created in a controlled environment. The part is transferred into the vacuum chamber and the metals are very accurately deposited onto the substrate. Titanium Nitride is a surface treatment that extends the life of high steel and carbide metal cutting tools.

In another example, painting is commonly specified by engineers to enhance a scaffold's appearance and corrosion resistance. Spray painting, electrostatic painting, dipping, brushing, and powder coat painting methods are some of the most common techniques used to apply the paint to the surface of the component. There are many types of paint formulations to protect metal parts in a wide range of physical environments. The automotive industry has automated the process of painting cars and trucks, utilizing thousands of robot arms and producing extremely consistent results.

In another example, thermal spraying is a type of surface treatment which involves melted or heated materials that are accelerated, then collide and bond mechanically to the target surface. A wire or powder feedstock, usually metal or ceramic, is melted by injecting it into a flame, electrical arc, or plasma stream. Engineers sometimes specify this process when added friction is a desirable characteristic. This technique is also commonly used on larger structural objects for protection against high temperatures, such as a thermal barrier coating for exhaust heat management.

In another example, acid etching involves a cutting of a hard surface such as metal or glass by a corrosive chemical, usually an acid. Acid etching of dental enamel with an acid in order to roughen the surface, increase retention of resin sealant, and promote mechanical retention are typical uses.

It will be appreciated that there are many other proprietary surface treatments and variants of the most common processes, which are engineered to improve or modify the characteristics of metallic parts.

In one embodiment or example, the catalyst particles are formed from a catalyst material or a catalyst supported material comprising the catalyst material on a support material. In an embodiment or example, the catalyst particles may be in a range from 0.1 nm to 10 mm, 1 nm to 5 mm, 100 nm to 1 mm, 1 μm to 200 μm, 0.25 μm to 50 μm, or 0.5 μm to 5 μm. The catalyst particles may be less than 5 mm, 1 mm, 100 μm, 10 μm, 5 μm, 1 μm, 500 nm, 250 nm, 100 nm, or 50 nm. The catalyst parties may be may at least 0.1 nm, 1 nm, 10 nm, 100 nm, 250 nm, 1 μm, 2 μm, 3 μm, 4 μm, 5 μm, 10 μm, 15 μm, 20 μm, 25 μm, or 50 μm. The catalyst particles may be a range provided by any two of these upper and/or lower values. It will be appreciated that the size of the catalyst particles are controlled by dry or wet milling if required. For example, the catalyst particles are less than 5 μm. It will also be appreciated that the catalyst particles formed from the catalyst material or the catalyst supported material are controlled by dry or wet milling if required. For example, the catalyst material or the catalyst supported material are less than 5 μm. In an embodiment or example, the catalyst particles may be in the form of spheres, pellets, cylinders, trilobes, honeycomb, plate, and quadralobes.

In another embodiment or example, the catalyst particles may be ex-situ catalyst particles. For example, an ex-situ catalyst may include a catalyst prepared such that the catalyst particles are in their final form prior to deposition to a surface of a scaffold. The catalyst particles in the catalytic liquid may be considered ex-situ if they have at least some catalytic activity irrespective of any activation reaction such as calcination. It will be appreciated that an ex-situ catalyst may differ from an in-situ catalyst. An in-situ catalyst may be a catalyst precursor applied to a surface of a scaffold which requires a further treatment step to produce the active catalyst in its final form, e.g. via activation such as calcination.

In another embodiment or example, the catalyst material can be selected from a metal, metal oxide, aluminium silicate, activated carbon, mesoporous carbon, graphene, graphitic material, metal-organic framework, zeolite, or any combination thereof. The catalyst material may be a metal, metal oxide or zeolite. In an example, the metal is selected from at least one of aluminium, iron, calcium, magnesium, cerium, cobalt, copper, zinc, nickel, palladium, platinum, gold, silicon, silver, ruthenium, iridium, rhodium, titanium, vanadium, zirconium, niobium, tantalum, and chromium, or a metal oxide thereof.

In another embodiment or example, the support material can be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide. The support material may be aluminium oxide, activated carbon, silica, zeolite, or calcium carbonate.

In another embodiment or example, the catalyst supported material can be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite. The catalyst supported material may be silica diphenyl phosphine palladium, zeolite, activated carbon, palladium on titanium silicate, lead-poisoned palladium on calcium carbonate, nickel modified aluminium oxide silicon oxide, or ruthenium on aluminium oxide.

The catalytic liquid suspension may comprise a catalyst material or a catalyst supported material. The catalyst particles may be formed from a catalyst material or a catalyst supported material. The catalyst supported material provides a catalyst material present with a support material. For example, a catalyst supported material may comprise catalyst particles containing catalyst material particles present with or on support material particles. It will be appreciated that when a catalyst material is used as a support material, the catalyst material and support material would be different. In one example, the support material may be provided as the major component of the catalyst particles, such as where support material particles are substantially larger in size than catalyst material particles that are present on the surface of the support material particles.

In an embodiment or example, the concentration of the catalyst material in the catalytic liquid suspension may be less than 20 wt. %, 15 wt. %, 10 wt. %, 8 wt. %, 6 wt. %, 5 wt. %, 4 wt. %, 3 wt. % or 2 wt. %. The concentration of the catalyst material in the catalytic liquid suspension may be at least 1 wt. %, 2 wt. %, 3 wt. %, 4 wt. %, 5 wt. %, 6 wt. %, 8 wt. %, 10 wt. %, or 15. wt %. The concentration of the catalyst material in the catalytic liquid suspension may be in the range of 1 to 20 wt. %, 2 to 15 wt. %, 3 to 10 wt. %, 4 to 8 wt. % or 5 to 6 wt. %. The concentration of the catalyst material may be a range provided by any two of these upper and/or lower values.

The weight % of the coating or catalyst material, based on total weight of catalytic static mixer, may be in the range of 1 to 40%, 2 to 35%, 5 to 30%, 10 to 25%, or 15 to 20%. The weight % of the coating comprising the catalyst material, based on total weight of catalytic static mixer, may be at least 1%, 5%, 10%, 15%, 20%, 35%, 30%, 35%, or 40%. The weight % of the coating comprising the catalyst material, based on total weight of catalytic static mixer, may be less than 50%, 40%, 30%, 20%, 15%, 10%, 5%, or 3%. The weight % of the coating comprising the catalyst material may be a range provided by any two of these upper and/or lower values.

In an embodiment or example, the binder can be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide or polyisobutylene. The binder may be hydroxypropyl cellulose, sucrose, dextrin, or starch. The binder may be hydroxypropyl cellulose, polyvinyl acetate, polyethylene glycol, sodium silicate or colloidal silica. The binder may be hydroxypropyl cellulose, polyvinyl acetate or colloidal silica. The binder may be hydroxypropyl cellulose, polyvinyl acetate or polydimethyl siloxane. The binder may be boehmite or colloidal aluminium oxide.

In an embodiment or example, the binder can be added at a concentration of between about 0.3 wt. % to about 5 wt. %. The concentration of the binder in the catalytic liquid suspension may be in a range of 0.1 wt. % to 10 wt. %, 0.2 wt. % to 8 wt. %, 0.3 wt. % to 6 wt. %, 0.4 wt. % to 5 wt. % or 0.5 wt. % to 3 wt. %. The concentration of the binder in the catalytic liquid suspension may be less than about 10 wt. %, 8 wt. %, 6 wt. %, 4 wt. %, 3 wt. %, 2 wt. %, 1 wt. %, 0.9 wt. %, 0.8 wt. %, 0.6 wt. %, 0.4 wt. %, 0.2 wt. % or 0.1 wt. %. The concentration of the binder in the catalytic liquid suspension may be at least about 0.1 wt. %, 0.2 wt. %, 0.3 wt. %, 0.4 wt. %, 0.5 wt. %, 0.6 wt. %, 0.7 wt. %, 0.8 wt. %, 0.9 wt. %, 1 wt. %, 1.5 wt. %, 2 wt. %, 2.5 wt. %, 3 wt. %, 3.5 wt. %, 4 wt. %, 4.5 wt. % or 5 wt. %. The concentration of the binder in the catalytic liquid suspension may be a range provided by any two of these upper and/or lower values.

In an embodiment or example, the liquid carrier may be selected from the group comprising water, ethanol, isopropanol, butanol, ethyl acetate, acetone or a combination thereof. For example, the solvent may be selected from water or ethanol. The solvent may be water. The solvent may be ethanol.

In an embodiment or example, the liquid suspension may have a solids content of between about 3 wt. % to about 28 wt. %. The solids content may be less than 28 wt. %, 27 wt. %, 25 wt. %, 23 wt. %, 21 wt. %, 19 wt. %, 17 wt. %, 15 wt. %, 13 wt. %, 11 wt. % 9 wt. %, 7 wt. %, 5 wt. %, or 3 wt. %. The solids content may be at least 4 wt. %, 6 wt. %, 8 wt. %, 10 wt. %, 12 wt. %, 14 wt. %, 16 wt. %, 18 wt. %, 20 wt. %, 22 wt. %, 24 wt. %, or 26 wt. %. The solids content may be a range provided by any two of these upper and/or lower values.

In an embodiment or example, the thickness of the coating is between about 1 μm to about 50 μm. The thickness of the coating may be in the range of 1 to 40%, 2 to 35%, 5 to 30%, 10 to 25%, or 15 to 20%. The thickness of the coating may be at least 1%, 5%, 10%, 15%, 20%, 35%, 30%, 35%, or 40%. The thickness of the coating may be less than 50%, 40%, 30%, 20%, 15%, 10%, 5%, or 3%. The thickness of the coating may be a range provided by any two of these upper and/or lower values.

In an embodiment or example, the surface area of the catalyst is between about 1 m²/g to about 1000 m²/g. The surface area of the catalyst may be at least 1, 5, 50, 100, 150, 200, 250, 300, 350, 400, 550, 600, 650, 700, 750, 800, 850, 900, 950 m²/g. The surface area of the catalyst may be in a range of about 5 to 200 m²/g, 100 to 250 m²/g, 150 to 1000 m²/g, or 200 to 800 m²/g. The surface area of the catalyst may be a range provided by any two of these upper and/or lower values.

It will be appreciated that the total mass loss of the coating may relate to the integrity of adhesion of the coatings. It will be appreciated that the adhesiveness of the coating may be determined via the mass loss from a sonication test. The inventors have surprisingly found that the process for coating a scaffold of a static mixer, as described here, advantageously provides a catalytic static mixer scaffold with enhanced or improved coating adhesiveness. In an embodiment or example, the total mass loss of the coating may be less than about 5 wt. %. The total mass loss of the coating may be less than about 5 wt. %, 4 wt. %, 3 wt. %, 2 wt. %, 1 wt. %, 0.8 wt. %, 0.6 wt. %, 0.4 wt. %, 0.2 wt. % or 0.1 wt. %. In an embodiment or example, the total mass loss of the coating may be less than about 0.5 wt. %. The total mass loss of the coating may be less than about 1 wt. %, 0.8 wt. %, 0.6 wt. %, 0.4 wt. %, 0.2 wt. % or 0.1 wt. %. It will be appreciated that adhesion is an important quality a coating, as it allows the coating to fulfil its function of protecting or decorating the substrate. It will be appreciated that various adhesion test methods can be applied to determine the adhesive strength of a coating on a scaffold as the force required to rupture the bonds between the coating and scaffold under prescribed conditions. For example, two different testing methods are covered by ASTM D3359-17 or ASTM D6677. The inventors have surprisingly found that the enhanced or improved coating adhesiveness prepared by the process, as described herein, advantageously provides an active catalyst metal on the catalytically active static mixer scaffold, that can be used continuously for prolonged periods of time with negligible degradation of the active catalyst metal. In an embodiment or example, the total leaching rate of the active catalyst metal under standard operation conditions may be a contamination of less than 350 parts-per-billion (ppb) in the reactor effluent. The total leaching rate may be less than about (ppb) 400, 350, 300, 250, 200, 150, 100, 70, 40, 10, 5, 1, or 0.5.

The catalytic liquid suspension may comprise or consist of a catalyst material or a catalyst supported material. The catalytic liquid suspension may comprise or consist of catalyst particles and a binder. The catalytic liquid suspension may comprise or consist a catalyst material, a support material, a binder, and a liquid carrier. The catalytic liquid suspension may comprise or consist a catalyst material, a binder, and a liquid carrier. The catalytic liquid suspension may comprise or consist a catalyst supported material, a binder, and a liquid carrier. The catalytic liquid suspension may comprise or consist a catalyst supported material, a binder, a support material and a liquid carrier. The catalytic liquid suspension may comprise or consist a catalyst material and a liquid carrier. The catalytic liquid suspension may comprise or consist a catalyst supported material and a liquid carrier. The catalytic liquid suspension may comprise or consist a catalyst supported material, a support material, and a liquid carrier.

### Option (a)

In an embodiment or example, there is provided a process for preparing a catalytically coated scaffold, comprising the step of: (i) applying a catalytic liquid suspension to a surface of a scaffold to provide a coating containing catalytically reactive sites on the surface of the scaffold, wherein the catalytic liquid suspension comprises a liquid carrier containing a plurality of catalyst particles of less than about 5 μm. For example, the surface of the scaffold is a static mixer. The catalytic liquid suspension comprising the liquid carrier containing a plurality of catalyst particles of less than about 5 μm may comprise or consist a catalyst material or a catalyst supported material. The catalytic liquid suspension may comprise or consist a catalyst material and a catalyst supported material. The catalytic liquid suspension may comprise or consist a catalyst material. The catalytic liquid suspension may comprises or consist a catalyst supported material. The catalytic liquid suspension may comprises or consist a catalyst material and a catalyst supported material. The catalytic liquid suspension may comprise or consist a catalyst material and a liquid carrier. The catalytic liquid suspension may comprise or consist a catalyst supported material and a liquid carrier.

In another embodiment or example, the catalyst material can be selected from a metal, metal oxide, aluminium silicate, activated carbon, mesoporous carbon, graphene, graphitic material, metal-organic framework, zeolite, or any combination thereof. The catalyst material may be a metal, metal oxide or zeolite. In an example, the metal is selected from at least one of aluminium, iron, calcium, magnesium, cerium, cobalt, copper, zinc, nickel, palladium, platinum, gold, silicon, silver, ruthenium, iridium, rhodium, titanium, vanadium, zirconium, niobium, tantalum, and chromium, or a metal oxide thereof. In an embodiment or example, the catalyst supported material can be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite. The catalyst supported material may be silica diphenyl phosphine palladium, zeolite, activated carbon, palladium on titanium silicate, lead-poisoned palladium on calcium carbonate, nickel modified aluminium oxide silicon oxide, or ruthenium on aluminium oxide. In an embodiment or example, the catalyst material or catalyst supported material may be activated charcoal, ruthenium on aluminium oxide, palladium on aluminium oxide, silica diphenyl phosphine palladium, palladium on titanium silicate, mesoporous carbon, zeolite, or nickel modified aluminium oxide silicon oxide. The catalyst material may be ruthenium on aluminium oxide, palladium on aluminium oxide, silver on aluminium oxide, or iron on aluminium oxide. The catalyst supported material may be ruthenium on aluminium oxide or palladium on aluminium oxide. In an embodiment or example, the liquid carrier may be ethanol or water. In an example, the catalyst supported material may be ruthenium on aluminium oxide and the liquid carrier may be water. In another example, the catalyst supported material may be palladium on aluminium oxide and the liquid carrier may be water. In another example, the catalyst supported material may be silver on aluminium oxide and the liquid carrier may be water. In another example, the catalyst supported material may be iron on aluminium oxide and the liquid carrier may be water. In an example, the catalyst supported material may be ruthenium on aluminium oxide and the liquid carrier may be ethanol. In another example, the catalyst supported material may be palladium on aluminium oxide and the liquid carrier may be ethanol. In another example, the catalyst supported material may be silver on aluminium oxide and the liquid carrier may be ethanol. In another example, the catalyst supported material may be iron on aluminium oxide and the liquid carrier may be ethanol.

### Option (b)

In an embodiment or example, there is provided a process for preparing a catalytically coated scaffold, comprising the step of: (i) applying a catalytic liquid suspension to a surface of a scaffold to provide a coating containing catalytically reactive sites on the surface of the scaffold, wherein the catalytic liquid suspension comprises a liquid carrier containing a plurality of catalyst particles of less than about 5 μm. For example, the surface of the scaffold is a static mixer comprising a support material.

The catalytic liquid suspension comprising a liquid carrier containing a plurality of catalyst particles of less than about 5 μm may comprises or consist a catalyst material and a support material. The catalytic liquid suspension may comprise or consist a catalyst supported material and a support material. The catalytic liquid suspension may comprises or consist a catalyst supported material, a support material and a liquid carrier. The catalytic liquid suspension may comprises or consist a catalyst material, a support material, and a liquid carrier.

In another embodiment or example, the catalyst material can be selected from a metal, metal oxide, aluminium silicate, activated carbon, mesoporous carbon, graphene, graphitic material, metal-organic framework, zeolite, or any combination thereof. The catalyst material may be a metal, metal oxide or zeolite. In an example, the metal is selected from at least one of aluminium, iron, calcium, magnesium, cerium, cobalt, copper, zinc, nickel, palladium, platinum, gold, silicon, silver, ruthenium, iridium, rhodium, titanium, vanadium, zirconium, niobium, tantalum, and chromium, or a metal oxide thereof. For example, the metal oxide may be iron oxide, cerium oxide, manganese oxide, vanadium oxide, or cobalt oxide. In another embodiment or example, the catalyst supported material can be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite. The catalyst supported material may be silica diphenyl phosphine palladium, zeolite, activated carbon, palladium on titanium silicate, lead-poisoned palladium on calcium carbonate, nickel modified aluminium oxide silicon oxide, or ruthenium on aluminium oxide. The catalyst supported material may be silica diphenyl phosphine palladium, zeolite, mesoporous carbon, palladium on titanium silicate, lead-poisoned palladium on calcium carbonate, nickel modified aluminium oxide silicon oxide, or ruthenium on aluminium oxide. In an embodiment or example, the catalyst material or catalyst supported material may be silica diphenyl phosphine palladium or zeolite. In an embodiment or example, the support material can be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide. The support material may be aluminium oxide, activated carbon, silica, zeolite, or calcium carbonate. In an embodiment or example, the support material may be aluminium oxide. In an embodiment or example, the liquid carrier may be ethanol or water. In an example, the catalyst supported material may be silica diphenyl phosphine palladium, the support material may be aluminium oxide, and the liquid carrier may be ethanol. In an example, the catalyst supported material may be silica diphenyl phosphine palladium, the support material may be aluminium oxide, and the liquid carrier may be water. In another example, the catalyst material may be selected from iron oxide, cerium oxide, manganese oxide, vanadium oxide, or cobalt oxide, the support material may be aluminium oxide, and the liquid carrier may be water or ethanol. In another example, the catalyst material may be iron oxide, the support material may be aluminium oxide, and the liquid carrier may be water. In another example, the catalyst material may be iron oxide, the support material may be aluminium oxide, and the liquid carrier may be ethanol. In another example, the catalyst material may be cerium oxide, the support material may be aluminium oxide, and the liquid carrier may be water. In another example, the catalyst material may be cerium oxide, the support material may be aluminium oxide, and the liquid carrier may be ethanol. In another example, the catalyst material may be manganese oxide, the support material may be aluminium oxide, and the liquid carrier may be water. In another example, the catalyst material may be manganese oxide, the support material may be aluminium oxide, and the liquid carrier may be ethanol. In another example, the catalyst material may be vanadium oxide, the support material may be aluminium oxide, and the liquid carrier may be water. In another example, the catalyst material may be vanadium oxide, the support material may be aluminium oxide, and the liquid carrier may be ethanol. In another example, the catalyst material may be cobalt oxide, the support material may be aluminium oxide, and the liquid carrier may be ethanol. In another example, the catalyst material may be cobalt oxide, the support material may be aluminium oxide, and the liquid carrier may be water.

### Option (c)

In an embodiment or example, there is provided a process for preparing a catalytically coated scaffold, comprising the step of: (i) applying a catalytic liquid suspension to a surface of a scaffold to provide a coating containing catalytically reactive sites on the surface of the scaffold, wherein the catalytic liquid suspension comprises a liquid carrier containing a plurality of catalyst particles of less than about 5 μm. For example, the surface of the scaffold is a static mixer comprising a support material and a binder.

The catalytic liquid suspension comprising a liquid carrier containing a plurality of catalyst particles of less than about 5 μm may comprise or consist a catalyst material, a support material, a binder, and a liquid carrier. The catalytic liquid suspension may comprise or consist a catalyst supported material, a support material, a binder, and a liquid carrier.

In another embodiment or example, the catalyst material can be selected from a metal, metal oxide, aluminium silicate, activated carbon, mesoporous carbon, graphene, graphitic material, metal-organic framework, zeolite, or any combination thereof. The catalyst material may be a metal, metal oxide or zeolite. In an example, the metal is selected from at least one of aluminium, iron, calcium, magnesium, cerium, cobalt, copper, zinc, nickel, palladium, platinum, gold, silicon, silver, ruthenium, iridium, rhodium, titanium, vanadium, zirconium, niobium, tantalum, and chromium, or a metal oxide thereof. In another embodiment or example, the catalyst supported material can be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite. The catalyst supported material may be silica diphenyl phosphine palladium, zeolite, activated carbon, palladium on titanium silicate, lead-poisoned palladium on calcium carbonate, nickel modified aluminium oxide silicon oxide, or ruthenium on aluminium oxide. In an embodiment or example, the catalyst material or catalyst supported material may be zeolite or activated carbon. In another embodiment or example, the catalyst supported material may be zeolite. In an embodiment or example, the support material can be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide. The support material may be aluminium oxide, activated carbon, silica, zeolite, or calcium carbonate. The support material may be aluminium oxide. In an embodiment or example, the binder can be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide or polyisobutylene. The binder may be hydroxypropyl cellulose, sucrose, dextrin, or starch. The binder may be hydroxypropyl cellulose, polyvinyl acetate, polyethylene glycol, sodium silicate or colloidal silica. The binder may be hydroxypropyl cellulose, polyvinyl acetate or colloidal silica. The binder may be hydroxypropyl cellulose, polyvinyl acetate or polydimethyl siloxane. The binder may be boehmite or colloidal aluminium oxide. The binder may be hydroxypropyl cellulose or colloidal silica. In an embodiment or example, the binder may be hydroxypropyl cellulose. In an embodiment or example, the liquid carrier may be ethanol or water. In an example, the catalyst material may be zeolite, the support material may be aluminium oxide, the binder may be hydroxypropyl cellulose, and the liquid carrier may be water. In an example, the catalyst material may be zeolite, the support material may be aluminium oxide, the binder may be hydroxypropyl cellulose, and the liquid carrier may be ethanol. In an example, the catalyst material may be zeolite, the support material may be aluminium oxide, the binder may be hydroxypropyl cellulose, and the liquid carrier may be ethanol. In an example, the catalyst material may be zeolite, the support material may be aluminium oxide, the binder may be colloidal silica, and the liquid carrier may be ethanol. In an example, the catalyst material may be zeolite, the support material may be aluminium oxide, the binder may be colloidal silica, and the liquid carrier may be water. In an example, the catalyst material may be activated carbon, the support material may be aluminium oxide, the binder may be colloidal silica, and the liquid carrier may be water. In an example, the catalyst material may be activated carbon, the support material may be aluminium oxide, the binder may be colloidal silica, and the liquid carrier may be ethanol. In an example, the catalyst material may be activated carbon, the support material may be aluminium oxide, the binder may be hydroxypropyl cellulose, and the liquid carrier may be ethanol. In an example, the catalyst material may be activated carbon, the support material may be aluminium oxide, the binder may be hydroxypropyl cellulose, and the liquid carrier may be water.

### Option (d)

In an embodiment or example, there is provided a process for preparing a catalytically coated scaffold, comprising the step of: (i) applying a catalytic liquid suspension to a surface of a scaffold to provide a coating containing catalytically reactive sites on the surface of the scaffold, wherein the catalytic liquid suspension comprises a liquid carrier containing a plurality of catalyst particles of less than about 5 μm. For example, the surface of the scaffold is a static mixer wherein the catalytic liquid suspension comprises a liquid carrier containing a plurality of catalyst particles of less than about 5 μm and a binder.

The catalytic liquid suspension comprising a liquid carrier containing a plurality of catalyst particles of less than about 5 μm may comprise or consist a catalyst material, a binder, and a liquid carrier. The catalytic liquid suspension may comprise or consist a catalyst supported material, a binder, and a liquid carrier.

In another embodiment or example, the catalyst material can be selected from a metal, metal oxide, aluminium silicate, activated carbon, mesoporous carbon, graphene, graphitic material, metal-organic framework, zeolite, or any combination thereof. The catalyst material may be a metal, metal oxide or zeolite. In an example, the metal may be selected from at least one of aluminium, iron, calcium, magnesium, cerium, cobalt, copper, zinc, nickel, palladium, platinum, gold, silicon, silver, ruthenium, iridium, rhodium, titanium, vanadium, zirconium, niobium, tantalum, and chromium, or a metal oxide thereof. In another embodiment or example, the catalyst supported material can be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite. The catalyst supported material may be silica diphenyl phosphine palladium, zeolite, activated carbon, palladium on titanium silicate, lead-poisoned palladium on calcium carbonate, nickel modified aluminium oxide silicon oxide, or ruthenium on aluminium oxide. In an embodiment or example, the binder can be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide or polyisobutylene. The binder may be hydroxypropyl cellulose, sucrose, dextrin, or starch. The binder may be hydroxypropyl cellulose, polyvinyl acetate, polyethylene glycol, sodium silicate or colloidal silica. The binder may be hydroxypropyl cellulose, polyvinyl acetate or polydimethyl siloxane. The binder may be boehmite or colloidal aluminium oxide. The binder may be hydroxypropyl cellulose. The binder may be hydroxypropyl cellulose, colloidal silica, polyvinylidene fluoride, poly(acrylic acid) sodium salt, or polyvinyl acetate. In an embodiment or example, the liquid carrier may be ethanol or water. In an embodiment or example, the catalyst supported material may be silica diphenyl phosphine palladium, lead-poisoned palladium on calcium carbonate, palladium on titanium silicate, nickel modified aluminium oxide silicon oxide, mesoporous carbon, or zeolite, the binder may be hydroxypropyl silica or colloidal silica, and the liquid carrier may be ethanol or water. In an example, the catalyst supported material may be ruthenium on aluminium, the binder may be hydroxypropyl cellulose, and the liquid carrier may be ethanol. In another example, the catalyst supported material may be lead-poisoned palladium on calcium carbonate, the binder may be hydroxypropyl cellulose, and the liquid carrier may be ethanol. In another example, the catalyst supported material may be palladium on titanium silicate, the binder may be hydroxypropyl cellulose, and the liquid carrier may be ethanol. In another example, the catalyst supported material may be palladium on titanium silicate, the binder may be hydroxypropyl cellulose, and the liquid carrier may be water. In another example, the catalyst supported material may be nickel modified aluminium oxide silicon oxide, the binder may be hydroxypropyl cellulose, and the liquid carrier may be ethanol. In another example, the catalyst material may be mesoporous carbon, the binder may be hydroxypropyl cellulose, and the liquid carrier may be ethanol. In another example, the catalyst material may be zeolite, the binder material may be hydroxypropyl cellulose, and the liquid carrier may be water. In another example, the catalyst material may be zeolite, the binder may be silicon oxide, and the liquid carrier may be water. In another example, the catalyst material may be zeolite, the binder material may be hydroxypropyl cellulose and silicon oxide, and the liquid carrier may be water. In an example, the catalyst supported material may be ruthenium on aluminium, the binder may be colloidal silica, and the liquid carrier may be ethanol. In another example, the catalyst supported material may be lead-poisoned palladium on calcium carbonate, the binder may be colloidal silica, and the liquid carrier may be ethanol. In another example, the catalyst supported material may be palladium on titanium silicate, the binder may be colloidal silica, and the liquid carrier may be ethanol. In another example, the catalyst supported material may be palladium on titanium silicate, the binder may be colloidal silica, and the liquid carrier may be water. In another example, the catalyst supported material may be nickel modified aluminium oxide silicon oxide, the binder may be colloidal silica, and the liquid carrier may be ethanol. In another example, the catalyst material may be mesoporous carbon, the binder may be colloidal silica, and the liquid carrier may be ethanol. In another example, the catalyst material may be zeolite, the binder material may be colloidal silica, and the liquid carrier may be water. In another example, the catalyst material may be zeolite, the binder may be colloidal silica, and the liquid carrier may be water. In another example, the catalyst material may be zeolite, the binder material may be colloidal silica, and the liquid carrier may be water. In another example, the catalyst material may be calcium oxide, the binder may be hydroxypropyl cellulose, and the liquid carrier may be ethanol. In another example, the catalyst material may be magnesium oxide, the binder may be hydroxypropyl cellulose, and the liquid carrier may be ethanol.

### Scaffolds

In an embodiment or example, the scaffold may be applied to any device or apparatus. In another embodiment or example, the scaffold may be a complex 3D structure. The complex 3D structure may be porous. In an embodiment or example, the scaffold may be suitable for continuous flow processes. The process described herein may be suitable for coating internal surfaces of a continuous flow reactor. In an embodiment or example, the scaffold may be a rotating mixer, a continuous pipe, a micromixer, a static mixer, integral porous insert, a metal foam, structured metallic or ceramic materials with voids such as honeycomb, metal mesh/plate, templated structure, or packed bed systems. In an embodiment or example, the scaffold may be a static mixer.

The scaffold of the static mixer may comprise or consist one of at least one of a metal, metal alloy, cermet, silicon carbide, glass, ceramic, mineral (e.g. calcium phosphate), or polymer. The scaffold may be a metal scaffold, for example formed from metals or metal alloys. The metal scaffold may be prepared from a material suitable for additive manufacturing (i.e. 3D printing). The metal scaffold may be prepared from a material suitable for further surface modification to provide or enhance catalytic reactivity, for example a metal including nickel, titanium, palladium, platinum, gold, copper, aluminium or their alloys and others, including metal alloys such as stainless steel. In one embodiment the metal for the scaffold may comprise or consist of titanium, stainless steel, and an alloy of cobalt and chromium. In another embodiment, the metal for the scaffold may comprise or consist of titanium, aluminium or stainless steel. In another embodiment, the metal for the scaffold may comprise or consist of stainless steel and cobalt chromium alloy. Using additive manufacturing techniques, i.e. 3D metal printing, the metal scaffold can be specifically designed to perform two major tasks: a) to act as a catalytic layer or a substrate for a catalytic layer, b) to act as a flow guide for optimal mixing performance during the chemical reaction and subsequently assist transfer of exothermic heat to the walls of the reactor tube (single phase liquid stream or multiphase stream) inside the reactor. Alternatively, the scaffold can be made from carbon fibre. Alternatively, the scaffold can be made from a glass. Alternatively, the scaffold can be made from a ceramic. Alternatively, the scaffold can be made from a mineral (e.g. calcium phosphate such as hydroxyapatite). Alternatively, the scaffold may be made from a polymer. The polymer may be thermoset or thermoplastic. Examples of polymers which can be used include, but are not limited to: polycarbonate, polymethylmethacrylate, polypropylene, polyethylene, polyamide, polyacrylamide, polyvinylchloride, or copolymers or any combinations thereof.

A catalyst material or catalyst supported material may refer to a catalyst by itself or to a material or composition comprising a catalyst. The catalyst material or catalyst supported material may be provided in a composition with one or more additives, such as binders, to facilitate coating of the catalyst to the scaffold. The catalyst or coating thereof may be provided as a partial coating or a complete layer on the scaffold. The coating or layer of the catalyst on the scaffold may be provided in one or more layers. The catalyst may be deposited on the scaffold by wash-coating or dip-coating. In an embodiment or example, the coating or layer of the catalyst may be a plurality of catalyst layers. For example, the coating or layer of the catalyst may be one of more layers. The coating or layer of the catalyst may be 2 to 10 layers. The coating or layer of the catalyst may be at least 2, 3, 4, 5, 6, 7, or 8 layers. The coating or layer of the catalyst may be less than 8, 7, 6, 5, 4, 3, or 2 layers. The coating or layer of the catalyst may be a range provided by any two of these upper and/or lower values.

In an embodiment or example, the catalyst loading may be less than 20 wt. %, 18 wt. %, 16 wt. %, 14 wt. %, 12 wt. %, 10 wt. %, 8 wt. %, 6 wt. %, 4 wt. %, or 2 wt. %. The catalyst loading may be at least 1 wt. %, 3 wt. %, 5 wt. %, 7 wt. %, 9 wt. %, 11 wt. %, 13 wt. %, 15 wt. %, 17 wt. % or 19 wt. %. The catalyst loading may be a range provided by any two of these upper and/or lower values.

In one embodiment, the scaffold may be a metal scaffold comprising a coating comprising catalyst material. It will be appreciated that a metal scaffold comprising a coating comprising catalyst material may be referred to as a catalytically coated scaffold. In another embodiment, the metal scaffold comprises titanium, nickel, aluminium, stainless steel, cobalt, chromium, any alloy thereof, or any combination thereof. In another embodiment, the metal scaffold comprises at least one of a stainless steel and aluminium. In another embodiment, the metal scaffold comprises titanium, or a titanium alloy. Further advantages may be provided wherein the metal scaffold comprises or consists of stainless steel or a cobalt chromium alloy.

The weight % of the coating or catalyst material, based on total weight of catalytic static mixer, may be in the range of 1 to 40%, 2 to 35%, 5 to 30%, 10 to 25%, or 15 to 20%. The weight % of the coating comprising the catalyst material, based on total weight of catalytic static mixer, may be at least 1%, 5%, 10%, 15%, 20%, 35%, 30%, 35%, or 40%. The weight % of the coating comprising the catalyst material, based on total weight of catalytic static mixer, may be less than 50%, 40%, 30%, 20%, 15%, 10%, 5%, or 3%. The weight % of the coating or catalyst material, based on total weight of catalytic static mixer may be a range provided by any two of these upper and/or lower values.

In an embodiment or example, a catalytically coated scaffold comprising a coating on a scaffold may comprise a coating comprising a plurality of catalyst particles. In an embodiment or example, the coating comprises a support material and optionally a binder. The catalyst particles may be selected from a metal, metal oxide, aluminium silicate, activated carbon, mesoporous carbon, graphene, graphitic material, metal-organic framework, zeolite, or any combination thereof.

In an embodiment or example, the catalyst particles may be a metal selected from at least one of aluminium, iron, cerium, calcium, cobalt, copper, magnesium, zinc, nickel, palladium, platinum, gold, silicon, silver, ruthenium, iridium, rhodium, titanium, vanadium, zirconium, niobium, tantalum, and chromium, or a metal oxide thereof.

In an embodiment or example, the support material may be selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide.

In an embodiment or example, the catalyst supported material may be selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite.

In an embodiment or example, the binder may be selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide, or polyisobutylene.

In one embodiment or example, the catalyst particles are formed from a catalyst material or a catalyst supported material comprising the catalyst material on a support material. In an embodiment or example, the catalyst particles may be in a range from 0.1 nm to 10 mm, 1 nm to 5 mm, 100 nm to 1 mm, 1 μm to 200 μm, 0.25 μm to 50 μm, or 0.5 μm to 5 μm. The catalyst particles may be less than 5 mm, 1 mm, 100 μm, 10 μm, 5 μm, 1 μm, 500 nm, 250 nm, 100 nm, or 50 nm. The catalyst parties may be may at least 0.1 nm, 1 nm, 10 nm, 100 nm, 250 nm, 1 μm, 2 μm, 3 μm, 4 μm, 5 μm, 10 μm, 15 μm, 20 μm, 25 μm, or 50 μm. The catalyst particles may be a range provided by any two of these upper and/or lower values.

In an embodiment or example, the thickness of the coating may be between about 1 μm to about 50 μm. The thickness of the coating may be in the range of 1 to 40%, 2 to 35%, 5 to 30%, 10 to 25%, or 15 to 20%. The thickness of the coating may be at least 1%, 5%, 10%, 15%, 20%, 35%, 30%, 35%, or 40%. The thickness of the coating may be less than 50%, 40%, 30%, 20%, 15%, 10%, 5%, or 3%. The thickness of the coating may be a range provided by any two of these upper and/or lower values.

In an embodiment or example, the process further comprises a drying step. The drying step comprises the steps of: (a) applying a first temperature ranging between about 15° C. to about 30° C. to the coated surface of the scaffold for a first period of about 4 to 24 hours to volatilise at least a portion of volatile material from the catalytic liquid suspension; and (b) applying a second temperature ranging between about 100° C. to about 180° C. under reduced pressure for a second period of about 4 to 24 hours such that a dried coating is formed on the surface of the scaffold. The first temperature may be in range from 15° C. to 30° C. or 20° C. to 25° C. For example, the temperature may be ambient temperature. The first temperature may be less than 30° C., 28° C., 26° C., 24° C., 22° C., 20° C., 18° C., or 16° C. The first temperature may be at least 15° C., 17° C., 19° C., 21° C., 23° C., 25° C., or 27° C. The first temperature may be a range provided by any two of these upper and/or lower values. The first period may be in a range from 4 to 24 hours or 10 to 20 hours. The first period may be less than 30, 28, 26, 24, 22, 20, 18, 16, 14, 12, 10, or 8 hours. The first period may be at least 5, 7, 9, 11, 13, 15, 17, 19, 21, 23, or 25 hours. For example, the first period may be 24 hours. The first period may be a range provided by any two of these upper and/or lower values. The second temperature may be in range from 100° C. to 180° C. or 110° C. to 140° C. The second temperature may be less than 180° C., 170° C., 160° C., 140° C., 120° C., or 110° C. The second temperature may be at least 100° C., 105° C., 110° C., 115° C., 120° C., 125° C., or 130° C. For example the temperature may be 120° C. The second temperature may be a range provided by any two of these upper and/or lower values. The second period may be in a range from 4 to 24 hours or 10 to 20 hours. The second period may be less than 30, 28, 26, 24, 22, 20, 18, 16, 14, 12, 10, or 8 hours. The second period may be at least 5, 7, 9, 11, 13, 15, 17, 19, 21, 23, or 25 hours. The second period may be a range provided by any two of these upper and/or lower values.

The static mixer is for use in a continuous flow chemical reaction system and process. The process may be an in-line continuous flow process. The in-line continuous flow process may be a recycle loop or a single pass process. In one embodiment, the in-line continuous flow process is a single pass process.

As mentioned above, the chemical reactor comprising the static mixer scaffold is capable of performing heterogeneous catalysis reactions in a continuous fashion. The chemical reactor may use single or multi-phase feed and product streams. In one embodiment, the substrate feed (comprising one or more reactants) may be provided as a continuous fluidic stream, for example a liquid stream containing either: a) the substrate as a solute within an appropriate solvent, or b) a liquid substrate, with or without a co-solvent. It will be appreciated that the continuous fluidic stream may be provided by at least one liquid phase. It will be appreciated that the fluidic stream may be provided by one or more gaseous streams, for example a hydrogen gas or source thereof. The substrate feed is pumped into the reactor using pressure driven flow, e.g. by means of a piston pump.

The volume displacement % of the static mixer relative to a reactor chamber for containing the mixer is in the range of 1 to 40, 2 to 35, 3 to 30, 4 to 25, 5 to 20, or 10 to 15. The volume displacement % of the static mixer relative to a reactor chamber for containing the mixer may be less than 40%, 35%, 30%, 25%, 20%, 15%, 10%, or 5%.

The configurations of the static mixers may be provided to enhance cross-sectional microscopic turbulence. Such turbulence may result from various sources, including the geometry of CSM or the microscopic roughness of the CSM surface resulting from the 3D printing process. For example, turbulent length scales may be reduced to provide better mixing. The turbulent length scales may, for example, be in the microscopic length scales.

The configurations of the static mixers may be provided to enhance heat transfer properties in the reactor, for example a reduced temperature differential at the exit cross-section. The heat transfer of the CSM may, for example, provide a cross-sectional or transverse temperature profile that has a temperature differential of less than about 20° C./mm, 15° C./mm, 10° C./mm, 9° C./mm, 8° C./mm, 7° C./mm, 6° C./mm, 5° C./mm, 4° C./mm, 3° C./mm, 2° C./mm, or 1° C./mm.

The scaffold may be configured such that, in use, the pressure drop (or back pressure) across the static mixers (in Pa/m) is in a range of about 0.1 to 1,000,000 Palm (or 1 MPa/m), including at any value or range of any values therebetween. For example, the pressure drop (or back pressure) across the static mixer (in Pa/m) may be less than about 500,000, 250,000, 100,000, 50,000, 10,000, 5,000, 1,000, 750, 500, 250, 100, 75, 50, 25, 20, 15, 10, or 5 Pa/m. The static mixers may be configured to provide a lower pressure drop relative to a specific flow rate. In this regard, the static mixers, reactor, system, and processes, as described herein, may be provided with parameters suitable for industrial application. The above pressure drops may be maintained where the volumetric flow rate is at least 0.1, 0.5, 1, 1.5, 2, 2.5, 3, 3.5, 4, 4.5, 5, 6, 7, 8, 9, 10, 20, 30, 40, 50, 100, 500, 1000 ml/min.

The catalytic reactions may be hydrogen insertion reactions that involve the use of hydrogenation catalysts. A hydrogen insertion or hydrogenation catalyst facilitate the insertion of hydrogen into intramolecular bonds of a reactant, e.g., a carbon-oxygen bond to form the oxygen containing organic materials described above, conversion of unsaturated bonds to saturated bonds, removal of protection groups such as converting O-benzyl groups to hydroxyl groups, or reaction of a nitrogen triple bond to form ammonia or hydrazine or mixtures thereof. The hydrogen insertion or hydrogenation catalyst may be chosen from the group consisting of cobalt, ruthenium, osmium, nickel, palladium, platinum, and alloys, compounds and mixtures thereof. In an embodiment, the hydrogen insertion or hydrogenation catalyst comprises or consists of platinum or titanium. In ammonia synthesis the catalyst may facilitate the dissociative adsorption of a hydrogen species source and a nitrogen species source for subsequent reaction. In a further embodiment, the hydrogen insertion or hydrogenation catalyst is coated using wash-coating or dip-coating.

### Static Mixers

It will be appreciated that the static mixers can provide an integral scaffold for a chemical reactor chamber. The static mixer scaffold for a continuous flow chemical reactor chamber may comprise a catalytically active scaffold defining a plurality of passages configured for dispersing and mixing one or more fluidic reactants during flow and reaction thereof through the mixer. It will be appreciated that at least a substantial part of the surface of the scaffold may comprise a catalyst material. The catalyst material may be selected from at least one of a metal, metal oxide, aluminium silicate, activated carbon, mesoporous carbon, graphene, graphitic material, metal-organic framework, zeolite, for providing the surface of the static mixer scaffold with catalytically reactive sites.

The static mixer may be provided as one or more scaffolds each configured for inserting into a continuous flow chemical reactor or reactor chamber thereof. The static mixer scaffold may be configured as a modular insert for assembly into a continuous flow chemical reactor or chamber thereof. The static mixer scaffold may be configured as an insert for an in-line continuous flow chemical reactor or chamber thereof. The in-line continuous flow chemical reactor may be a recycle loop reactor or a single pass reactor. In one embodiment, the in-line continuous flow chemical reactor is a single pass reactor.

The static mixer scaffold may be configured for enhancing mixing and heat transfer characteristics for redistributing fluid in directions transverse to the main flow, for example in radial and tangential or azimuthal directions relative to a central longitudinal axis of the static mixer scaffold. The static mixer scaffold may be configured for at least one of (i) to ensure as much catalytic surface area as possible is presented to the flow so as to activate close to a maximum number of reaction sites and (ii) to improve flow mixing so that (a) the reactant molecules contact surfaces of the static mixer scaffold more frequently and (b) heat is transferred away from or to the fluid efficiently. The static mixer scaffold may be provided with various geometric configurations or aspect ratios for correlation with particular applications. The static mixer scaffolds enable fluidic reactants to be mixed and brought into close proximity with the catalytic material for activation. The static mixer scaffold may be configured for use with turbulent flow rates, for example enhancing turbulence and mixing, even at or near the internal surface of the reactor chamber housing. It will also be appreciated that the static mixer scaffold can be configured to enhance the heat and mass transfer characteristics for both laminar and turbulent flows.

The configurations may also be designed to enhance efficiency, degree of chemical reaction, or other properties such as pressure drop (whilst retaining predetermined or desired flow rates), residence time distribution or heat transfer coefficients. As previously mentioned, traditional static mixers have not been previously developed to specifically address enhanced heat transfer requirements, which may result from the catalytic reaction environments provided by the present static mixers.

The configuration of the scaffold, or static mixer, may be determined using Computational Fluid Dynamics (CFD) software, which can be used for enhancing the configuration for mixing of reactants for enhanced contact and activation of the reactants, or reactive intermediates thereof, at the catalytically reactive sites of the scaffold. The CFD based configuration determinations are described in further detail in sections below.

The static mixer scaffold may be formed by additive manufacturing. The static mixer may be an additive manufactured static mixer. Additive manufacturing of the static mixer and subsequent catalytic coating can provide a static mixer that is configured for efficient mixing, heat transfer and catalytic reaction (of reactants in continuous flow chemical reactors), and in which the static mixer may be physically tested for reliability and performance, and optionally further re-designed and re-configured using additive manufacturing (e.g. 3D printing) technology. Additive manufacturing provides flexibility in preliminary design and testing, and further re-design and re-configuration of the static mixers to facilitate development of more commercially viable and durable static mixers.

The static mixer scaffold may be provided in a configuration selected from one or more of the following general non-limiting example configurations:
open configurations with helices;
open configurations with blades;
corrugated-plates;
multilayer designs;
closed configurations with channels or holes;
irregular designs, such as metal foams or monoliths.

The scaffold of the static mixer may be provided in a mesh configuration having a plurality of integral units defining a plurality of passages configured for facilitating mixing of the one or more fluidic reactants.

The static mixer scaffold may comprise a scaffold provided by a lattice of interconnected segments configured to define a plurality of apertures for promoting mixing of fluid flowing through the reactor chamber. The scaffold may also be configured to promote both heat transfer as well as fluid mixing.

In various embodiments, the geometry or configuration may be chosen to enhance one or more characteristics of the static mixer scaffold selected from: the specific surface area, volume displacement ratio, strength and stability for high flow rates, suitability for fabrication using additive manufacturing, and to achieve one or more of: a high degree of chaotic advection, turbulent mixing, catalytic interactions, and heat transfer.

In some embodiments, the scaffold may be configured to enhance chaotic advection or turbulent mixing, for example cross-sectional, transverse (to the flow) or localised turbulent mixing. The geometry of the scaffold may be configured to change the localised flow direction or to split the flow more than a certain number of times within a given length along a longitudinal axis of the static mixer scaffold, such as more than 200 m⁻¹, optionally more than 400 m⁻¹, optionally more than 800 m⁻¹, optionally more than 1500 m⁻¹, optionally more than 2000 m⁻¹, optionally more than 2500 m⁻¹, optionally more than 3000 m⁻¹, optionally more than 5000 m⁻¹. The geometry or configuration of the scaffold may comprise more than a certain number of flow splitting structures within a given volume of the static mixer, such as more than 100 m⁻³, optionally more than 1000 m⁻³, optionally more than 1×10⁴ m⁻³, optionally more than 1×10⁶ m⁻³, optionally more than 1×10⁹ m⁻³, optionally more than 1×10¹⁰ m⁻³.

The geometry or configuration of the scaffold may be substantially tubular or rectilinear. The scaffold may be formed from or comprise a plurality of segments. Some or all of the segments may be straight segments. Some or all of the segments may comprise polygonal prisms such as rectangular prisms, for example. The scaffold may comprise a plurality of planar surfaces. The straight segments may be angled relative to each other. Straight segments may be arranged at a number of different angles relative to a longitudinal axis of the scaffold, such as two, three, four, five or six different angles, for example. The scaffold may comprise a repeated structure. The scaffold may comprise a plurality of similar structures repeated periodically along the longitudinal axis of the scaffold. The geometry or configuration of the scaffold may be consistent along the length of the scaffold. The geometry of the scaffold may vary along the length of the scaffold. The straight segments may be connected by one or more curved segments. The scaffold may comprise one or more helical segments. The scaffold may generally define a helicoid. The scaffold may comprise a helicoid including a plurality of apertures in a surface of the helicoid.

The dimensions of the static mixer may be varied depending on the application. The static mixer, or reactor comprising the static mixer, may be tubular. The static mixer or reactor tube may, for example, have a diameter (in mm) in the range of 1 to 5000, 2 to 2500, 3 to 1000, 4 to 500, 5 to 150, or 10 to 100. The static mixer or reactor tube may, for example, have a diameter (in mm) of at least about 1, 5, 10, 25, 50, 75, 100, 250, 500, or 1000. The static mixer or reactor tube may, for example, have a diameter (in mm) of less than about 5000, 2500, 1000, 750, 500, 250, 200, 150, 100, 75, or 50. The aspect ratios (L/d) of the static mixer scaffolds, or reactor chambers comprising the static mixer scaffolds, may be provided in a range suitable for industrial scale flow rates for a particular reaction. The aspect ratios may, for example, be in the range of about 1 to 1000, 2 to 750, 3 to 500, 4 to 250, 5 to 100, or 10 to 50. The aspect ratios may, for example, be less than about 1000, 750, 500, 250, 200, 150, 100, 75, 50, 25, 20, 15, 10, 9, 8, 7, 6, 5, 4, 3, or 2. The aspect ratios may, for example, be greater than about 1, 2, 3, 4, 5, 6, 7, 8, 9, 10, 15, 20, 25, 50, 75, or 100. It will be appreciated that the aspect ratio means the ratio of length to diameter (L/d) of a single unit or scaffold.

The static mixer scaffold or reactor is generally provided with a high specific surface area (i.e., the ratio between the internal surface area and the volume of the static mixer scaffold and reactor chamber). The specific surface area may be lower than that provided by a packed bed reactor system. The specific surface area (m² m⁻³) may be in the range of 100 to 40,000, 200 to 30,000, 300 to 20,000, 500 to 15,000, or 12000 to 10,000. The specific surface area (m² m⁻³) may be at least 100, 200, 300, 400, 500, 750, 1000, 2000, 3000, 4000, 5000, 7500, 10000, 12500, 15000, 17500, or 20000. It will be appreciated that the specific surface areas can be measured by a number of techniques including the BET isotherm techniques.

The static mixer scaffolds may be configured for enhancing properties, such as mixing and heat transfer, for laminar flow rates or turbulent flow rates. It will be appreciated that for Newtonian fluids flowing in a hollow pipe, the correlation of laminar and turbulent flows with Reynolds number (Re) values would typically provide laminar flow rates where Re is <2300, transient where 2300<Re<4000, and generally turbulent where Re is >4000. The static mixer scaffolds may be configured for laminar or turbulent flow rates to provide enhanced properties selected from one or more of mixing, degree of reaction, heat transfer, and pressure drop. It will be appreciated that further enhancing a particular type of chemical reaction will require its own specific considerations.

The static mixer scaffold may be generally configured for operating at a Re of at least 0.01, 0.1, 1, 5, 50, 100, 150, 200, 250, 300, 350, 400, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10000. The static mixer scaffold may be configured for operating in a generally laminar flow Re range of about 0.1 to 2000, 1 to 1000, 10 to 800, or 20 to 500. The static mixer scaffold may be configured for operating in a generally turbulent flow Re ranges of about 1000 to 15000, 1500 to 10000, 2000 to 8000, or 2500 to 6000.

The volume displacement % of the static mixer relative to a reactor chamber for containing the mixer is in the range of 1 to 40, 2 to 35, 3 to 30, 4 to 25, 5 to 20, or 10 to 15. The volume displacement % of the static mixer relative to a reactor chamber for containing the mixer may be less than 40%, 35%, 30%, 25%, 20%, 15%, 10%, or 5%.

The configurations of the static mixers may be provided to enhance cross-sectional microscopic turbulence. Such turbulence may result from various sources, including the geometry of CSM or the microscopic roughness of the CSM surface resulting from the 3D printing process and/or surface coating. For example, turbulent length scales may be reduced to provide better mixing. The turbulent length scales may, for example, be in the range of microscopic length scales.

The configurations of the static mixers may be provided to enhance heat transfer properties in the reactor, for example a reduced temperature differential at the exit cross-section. The heat transfer of the CSM may, for example, provide a cross-sectional or transverse temperature profile that has a temperature differential of less than about 20° C./mm, 15° C./mm, 10° C./mm, 9° C./mm, 8° C./mm, 7° C./mm, 6° C./mm, 5° C./mm, 4° C./mm, 3° C./mm, 2° C./mm, or 1° C./mm.

The scaffold may be configured such that, in use, the pressure drop (i.e. pressure differential or back pressure) across the static mixers (in Pa/m) is in a range of about 0.1 to 1,000,000 Pa/m (or 1 MPa/m), including at any value or range of any values therebetween. For example, the pressure drop across the static mixer (in Pa/m) may be less than about 500,000, 250,000, 100,000, 50,000, 10,000, 5,000, 1,000, 750, 500, 250, 100, 75, 50, 25, 20, 15, 10, or 5 Pa/m. The static mixers may be configured to provide a lower pressure drop relative to a specific flow rate. In this regard, the static mixers, reactor, system, and processes, as described herein, may be provided with parameters suitable for industrial application. The above pressure drops may be maintained where the volumetric flow rate is at least 0.1, 0.5, 1, 1.5, 2, 2.5, 3, 3.5, 4, 4.5, 5, 6, 7, 8, 9, 10, 20, 30, 40, 50 ml/min.

### Process for Preparing Static Mixer

The static mixer scaffold may be provided by additive manufacturing, such as 3D printing. Additive manufacturing of the static mixer and subsequent catalyst coating can provide a static mixer that is configured for efficient mixing, heat transfer and catalytic reaction (of reactants in continuous flow chemical reactors), and in which the static mixer may be physically tested for reliability and performance, and optionally further re-designed and re-configured using additive manufacturing (e.g. 3D printing) technology. Following original design and development using additive manufacturing, the static mixer may be prepared using other manufacturing process, such as casting (e.g. investment casting). The additive manufacturing provides flexibility in preliminary design and testing, and further re-design and re-configuration of the static mixers to facilitate development of more commercially viable and durable static mixers.

The static mixer scaffolds may be made by the additive manufacture (i.e. 3D printing) techniques. For example, an electron beam 3D printer or a laser beam 3D printer may be used. The additive material for the 3D printing may be, for example, titanium alloy based powders (e.g. 45-105 micrometre diameter range) or the cobalt-chrome alloy based powders (e.g. FSX-414) or stainless steel or aluminium-silicon alloy. The powder diameters associated with the laser beam printers are typically lower than those used with electron beam printers.

3D printing is well understood and refers to processes that sequentially deposit material onto a powder bed via fusion facilitated by the heat supplied by a beam, or by extrusion and sintering-based processes. 3D printable models are typically created with a computer aided design (CAD) package. Before printing a 3D model from an STL file, it is typically examined for manifold errors and corrections applied. Once that is done, the .STL file is processed by software called a “slicer,” which converts the model into a series of thin layers and produces a G-code file containing instructions tailored to a specific type of 3D printer. The 3D printing process is advantageous for use in preparing the static mixer scaffolds since it eliminates the restrictions to product design imposed by traditional manufacturing routes. Consequently, the design freedom inherited from 3D printing allows a static mixer geometry to be further optimised for performance than it otherwise would have been.

The catalytically active scaffold may be prepared from a catalyst material selected from at least one of a metal, metal oxide, aluminium silicate, activated carbon, mesoporous carbon, graphene, graphitic material, metal-organic framework, zeolite. The process of preparing a static mixer may comprise a step of applying a coating comprising the catalyst material onto at least a substantial portion of the scaffold by wash-coating or dip-coating. For example, the coating may be provided on at least 50% of the surface of the scaffold. In other embodiments, the coating may be provided on at least 60%, 70%, 80%, 90%, 95%, 98, or 99%, of the surface of the scaffold.

In some embodiments, the process may first comprise forming the scaffold using an additive manufacturing process, such as 3D printing.

### Examples

The present disclosure is further described by the following examples. It is to be understood that the following description is for the purpose of describing particular embodiments only and is not intended to be limiting with respect to the above description.

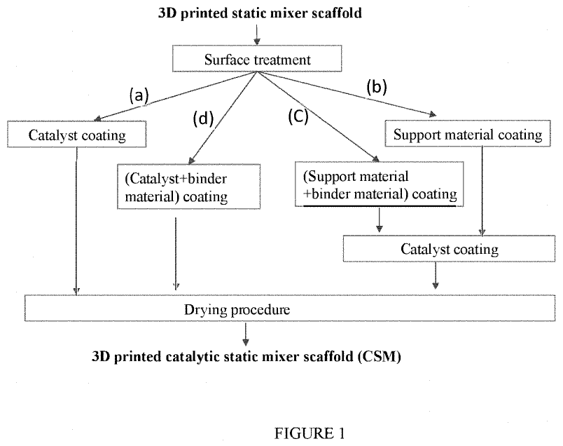

The present disclosure provides an effective and scalable process for the deposition of a catalyst material or catalyst supported material onto a static mixer scaffold by wash-coating/dip-coating the static mixer scaffold in a catalytic liquid suspension. Referring to FIG. 1, this process may include a number of different deposition processes, namely, option (a), option (b), option (c), and option (d) depending on the type of catalyst material or catalyst supported material required to be coated onto the static mixer scaffold.

It will be appreciated that the static mixer scaffold may be exposed to various optional surface pre-treatment methods to prepare the surface for the catalytic coating.

### Example 1 Optional Pre-Treatment Step

1) To etch the surface of the static mixer scaffold, the scaffold is first immersed into a diluted HCl solution (5-10 wt %) for 1 hour.
2) The scaffold is then soaked in sonication bath for 20 minutes.
3) The scaffold is immersed in a solution of acetone for 10 minutes.
4) The scaffold is the dried in an oven at 140° C. for 16 hours.

### Example 2 General Wash-Coating Steps for Coating Option (a)-(d)

### Option (a)

1) To form the catalytic liquid suspension, an amount of commercially sourced catalyst material or catalyst supported material with controlled particle size (typically <5 μm, by dry/wet milling if needed) is added into an amount of solvent and stirred on a magnetic stirrer over 48 hours to achieve homogenous slurry ca. 3-25 wt. % concentration of solid content.
2) The pre-treated static mixer scaffold is dipped into the suspension for 20-30 seconds, followed by pressurised air blowing to eliminate excess liquid and prevent channel blockage of the scaffold. The dipped scaffolds are then horizontally placed in an open container at room temperature for 1 day so evaporate any remaining solvent. Finally, the scaffolds are placed in an oven under 120° C. vacuum to eliminate the residue solvent. This process was repeated 2-6 times to gain more catalyst deposition layers onto catalytic static mixer scaffolds or until desired catalyst loading was achieved.

### Option (b)

1) To form the liquid support suspension support material into an amount of solvent, stirred on a magnetic stirrer over 24 hours to achieve homogenous slurry ca. 10-25 wt % concentration of solid content. The pre-treated static mixer scaffold was dipped into the liquid support suspension for 20-30 seconds, followed by pressurised air blowing to eliminate excess liquid and prevent channel blockage of the scaffold. The dipped scaffold was then horizontally placed in an open container at room temperature for 1 day so that most of the solvent would evaporate in the air. Then, the scaffold was placed in an oven under 120° C. vacuum to eliminate the residue solvent.
2) Step 1 forms the intermediate support layer before the coating of catalytic liquid suspension.
3) To form the catalytic liquid suspension, an amount of commercially sourced catalyst material or catalyst supported material with controlled particle size (typically <5 μm, by dry/wet milling if needed) is added into an amount of solvent and stirred on a magnetic stirrer over 48 hours to achieve homogenous slurry ca. 3-25 wt. % concentration of solid content.
4) The static mixer scaffold previously coated with an intermediate support layer was dipped into the suspension for 20-30 seconds, followed by pressurised air blowing to eliminate excess liquid and prevent channel blockage of the scaffold. The dipped scaffolds are then horizontally placed in an open container at room temperature for 1 day so evaporate any remaining solvent. Finally, the scaffolds are placed in an oven under 120° C. vacuum to eliminate the residue solvent. This process was repeated 2-6 times to gain more catalyst deposition layers onto catalytic static mixer scaffolds or until desired catalyst loading was achieved.

### Option (c)

1) To form the liquid support suspension support material and binder ca. 1 wt. % into an amount of solvent, stirred on a magnetic stirrer over 24 hours to achieve homogenous slurry ca. 10-25 wt % concentration of solid content. The pre-treated static mixer scaffold was dipped into the liquid support suspension for 20-30 seconds, followed by pressurised air blowing to eliminate excess liquid and prevent channel blockage of the scaffold. The dipped scaffold was then horizontally placed in an open container at room temperature for 1 day so that most of the solvent would evaporate in the air. Then, the scaffold was placed in an oven under 120° C. vacuum to eliminate the residue solvent.
2) Step 1 forms the intermediate support layer before the coating of catalytic liquid suspension.
3) To form the catalytic liquid suspension, an amount of commercially sourced catalyst material or catalyst supported material with controlled particle size (typically <5 μm, by dry/wet milling if needed) is added into an amount of solvent and stirred on a magnetic stirrer over 48 hours to achieve homogenous slurry ca. 3-25 wt. % concentration of solid content.
4) The static mixer scaffold previously coated with an intermediate support layer was dipped into the suspension for 20-30 seconds, followed by pressurised air blowing to eliminate excess liquid and prevent channel blockage of the scaffold.

The dipped scaffolds are then horizontally placed in an open container at room temperature for 1 day so evaporate any remaining solvent. Finally, the scaffolds are placed in an oven under 120° C. vacuum to eliminate the residue solvent. This process was repeated 2-6 times to gain more catalyst deposition layers onto catalytic static mixer scaffolds or until desired catalyst loading was achieved.

### Option (d)

1) To form the catalytic liquid suspension, an amount of commercially sourced catalyst material or catalyst supported material with controlled particle size (typically <5 μm, by dry/wet milling if needed) and a binder ca.1 wt. % is added into an amount of solvent and stirred on a magnetic stirrer over 48 hours to achieve homogenous slurry ca. 3-25 wt. % concentration of solid content.
2) The pre-treated static mixer scaffold is dipped into the suspension for 20-30 seconds, followed by pressurised air blowing to eliminate excess liquid and prevent channel blockage of the scaffold. The dipped scaffolds are then horizontally placed in an open container at room temperature for 1 day so evaporate any remaining solvent. Finally, the scaffolds are placed in an oven under 120° C. vacuum to eliminate the residue solvent. This process was repeated 2-6 times to gain more catalyst deposition layers onto catalytic static mixer scaffolds or until desired catalyst loading was achieved.

### Example 3 Adhesion Test

The solid content was checked by putting a few drops of the slurry onto a watch glass and drying it in an oven under 120° C. vacuum. The total solid content can be calculated with equation (1), whereas m₁ stands for the mass of the empty watch glass, m₂ stands for the mass of the watch glass added with the slurry and m₃ stands for the mass after evaporation of the solvent. The homogeneity of the slurry can also be inferred with several parallel solid content tests.

$$Total solid content wt \% = \frac{{m}_{3} - {m}_{1}}{{m}_{2} - {m}_{1}} ⨯ 100 \% \qquad ( 1 )$$

Before and after each coating step, the mass of the mixer was recorded to calculate the loading percentage with equation (2), where m₀ signifies the original mass of the mixer and mₙ signifies the mass after the nth coating.

$$Loading Percentage wt \% = \frac{{m}_{n} - {m}_{0}}{{m}_{0}} ⨯ 100 \% \qquad ( 2 )$$

To evaluate the adhesion of the catalyst layer, the coated CSMs were subjected to sonication in solvent bath e.g. water or ethanol for 10 min. The sonicated mixers were then horizontally placed in an open container at room temperature so that most of the solvent would evaporate in the air. Then, the mixers were placed in an oven under 120° C. vacuum to eliminate the residue solvent. After that, the mass loss was calculated using equation (3), where mn signifies the mass after the nth coating and ma signifies the mass after sonication.

$$Mass loss wt \% = - \frac{{m}_{a} - {m}_{n}}{{m}_{n}} ⨯ 100 \% \qquad ( 3 )$$

Table 1-4 provide examples of coated catalytic static mixer scaffold via different coating routes options (a)-(d). It will be appreciated that parallel experiments were done for each trial and data presented in the table are average values.

TABLE 1
Coated catalytic static mixer scaffolds via coating option (a).

| | | | | Binder | | | | Weight | |
|---|---|---|---|---|---|---|---|---|---|
| | | | | (b) | Solids | | Catalyst | after | Coating |
| | | Size | | Support | Content^{a} | Coatings | Loading | sonication | Success |
| Entry | Catalyst | (μm) | Solvent | (s) | (wt. %) | (n) | (wt. %) | (wt. %) | High/Low |
| 1 | Activated | 50 | water | — | 10 | 2 | Not tested^{b} | | Low |
| 2 | Charcoal | 35 | | — | 10 | 2 | | | Low |
| 3 | | 20 | | — | 10 | 2 | | | Low |
| 4 | | 5 | | — | 9 | 2 | 2.7 | 2.7 | Low |
| 5 | | 50 | ethanol | — | 11 | 2 | 2.6 | 2.6 | Low |
| 6 | Ru/Al₂O₃ | 4 | water | — | 15 | 2 | 2.8 | 0.3 | High |
| 7 | | | ethanol | — | 16 | 2 | 1.7 | 1.5 | Low |
| 8 | | | | | 22 | 2 | 4.8 | 4.6 | Low |
| 9 | Pd/Al₂O₃ | 2.5 | water | — | 24 | 4 | 5.0 | 0.5 | High |
| 10 | DPP-Pd | 0.25 | water | — | 16 | 2 | 0.5 | 0.5 | Low |
| 11 | | | ethanol | — | 13 | 1 | 0.1 | 0.1 | Low |
| 12 | Pd- | 3 | water | — | 16 | 2 | 1.7 | 0.7 | Low |
| | TiSiO₄ | | | | | | | | |
| 13 | Meso- | <0.5 | ethanol | — | Phase separation occurred | | | | Low |
| | porous | | | | | | | | |
| | Carbon | | | | | | | | |
| 14 | Zeolite | 4 | water | — | 25 | 2 | 3.6 | 2.8 | Low |
| 15 | Ni/Al₂O₃— | — | water | — | 22 | 2 | 4.8 | 2.9 | Low |
| 16 | SiO₂ | | ethanol | — | 23 | 2 | 2.8 | 1.5 | Low |

^{a}Solid content is the result of parallel experiments instead of the calculated value before mixing;
^{b}Coatings fell off unpreventably after drying.

TABLE 2
Coated catalytic static mixer scaffolds via coating option (b).

| | | | | Binder | | | | Weight | |
|---|---|---|---|---|---|---|---|---|---|
| | | Particle | | (b) | Solid | | Catalyst | after | Coating |
| | | Size | | Support | Content^{a} | Coating | Loading | sonication | Success |
| Entry | Catalyst | (μm) | Solvent | (s) | (wt. %) | (n) | (wt. %) | (wt. %) | High/Low |
| 1 | DPP-Pd | 0.25 | ethanol | s: 25 wt % | 24 | 3 | 3.7 | 0.2 | High |
| | | | | Al₂O₃ | | | | | |
| 2 | Zeolite | 4 | water | s: 20 wt % | 19 | 2 | 3.0 | 1.3 | Low |
| | | | | Al₂O₃ | | | | | |

^{a}Solid content is the result of parallel experiments instead of the calculated value before mixing.

TABLE 3
Coated catalytic static mixer scaffolds via coating option (c)

| | | | | Binder | | | | Weight | |
|---|---|---|---|---|---|---|---|---|---|
| | | Particle | | (b) | Solid | | Catalyst | after | Coating |
| | | Size | | Support | Content^{a} | Coatings | Loading | sonication | Success |
| Entry | Catalyst | (μm) | Solvent | (s) | (wt. %) | (n) | (wt. %) | (wt. %) | High/Low |
| 1 | Zeolite | 4 | water | s: 20 wt % | 20 | 2 | 5.2 | 0.6 | High |
| | | | | Al₂O₃ + | | | | | |
| | | | | b: 1 wt % | | | | | |
| | | | | HPC | | | | | |

^{a}Solid content is the result of parallel experiments instead of the calculated value before mixing.

TABLE 4
Coated CSM substrates via coating option (d)

| | | | | Binder | | | | Weight | |
|---|---|---|---|---|---|---|---|---|---|
| | | Particle | | (b) | Solid | | Catalyst | after | Coating |
| | | Size | | Support | Content^{a} | Coatings | Loading | sonication | Success |
| | Catalyst | (μm) | Solvent | (s) | (wt. %) | (n) | (wt. %) | (wt. %) | High/Low |
| 1 | Ru/Al₂O₃ | 4 | ethanol | b: 1 wt % | 22 | 1 | 5.8 | 0.6 | High |
| | | | | HPC | | | | | |
| 2 | | | | b: 0.5 wt % | 22 | 1 | 3.0 | 0.3 | High |
| | | | | HPC | | | | | |
| 3 | | | | b: 0.5 wt % | 10 | 2 | 1.7 | 0.2 | High |
| | | | | HPC | | | | | |
| 4 | | | | b: 0.25 wt % | 5 | 3 | 1.7 | 0.1 | High |
| | | | | HPC | | | | | |
| 5 | Activated | 5 | water | b: 0.5 wt % | 11 | 2 | 3.0 | 3.0 | Low |
| | Charcoal | | | HPC | | | | | |
| 6 | Pd-CaCO₃ | — | ethanol | b: 0.5 wt % | 24 | 6 | 5.0 | 0.5 | High |
| | Pb | | | HPC | | | | | |
| 7 | Pd-TiSiO₄ | 3 | water | b: 0.5 wt % | 16 | 2 | 2.3 | 0.3 | High |
| | | | | HPC | | | | | |
| 8 | | | | b: 0.5 wt % | 22 | 3 | 3.7 | 0.3 | High |
| | | | | HPC | | | | | |
| 9 | Ni/Al₂O₃— | — | ethanol | b: 0.5 wt % | 20 | 1 | 1.7 | 0.2 | High |
| | SiO₂ | | | HPC | | | | | |
| 10 | | | | b: 1 wt % | 20 | 2 | 7.5 | 0.1 | High |
| | | | | HPC | | | | | |
| 11 | Meso- | <0.5 | ethanol | b: 3.6 wt % | Phase separation occurred | | | | Low |
| | porous | | | PVA | | | | | |
| 12 | Carbon | | | b: 3.6 wt % | Phase separation occurred | | | | Low |
| | | | | NaPAA | | | | | |
| 13 | | | | b: 3.6 wt % | Phase separation occurred | | | | Low |
| | | | | PVDF | | | | | |
| 14 | | | | b: 1 wt % | 2.7 | 3 | 1.0 | 0.1 | High |
| | | | | HPC | | | | | |
| 15 | | | | b: 0.5 wt % | 3.4 | 3 | 1.7 | 1.5 | Low |
| | | | | HPC + | | | | | |
| | | | | 1.1 wt % | | | | | |
| | | | | CS | | | | | |
| 16 | | | | b: 1.1 wt % | 2.9 | 3 | 2.0 | 1.6 | Low |
| | | | | CS | | | | | |
| 17 | Pd/C¹ | — | ethanol | b: 0.9 wt % | 6.6 | 3 | 7.0 | 5.6 | Low |
| | | | | HPC + | | | | | |
| | | | | 0.2 wt % | | | | | |
| | | | | CS | | | | | |
| 18 | | | | b: 0.5 wt % | 5.4 | 3 | 8.9 | 6.5 | Low |
| | | | | HPC + | | | | | |
| | | | | 1.1 wt % | | | | | |
| | | | | CS | | | | | |
| 19 | Zeolite | 4 | water | b: 1 wt % | 26 | 2 | 4.8 | 0.9 | High |
| | | | | HPC | | | | | |
| 20 | | | | b: 3 wt % | 28 | 2 | 4.8 | 0.5 | High |
| | | | | SiO₂ | | | | | |
| 21 | | | | b: 3 wt % | 28 | 2 | 5.2 | 0.4 | High |
| | | | | SiO2 + | | | | | |
| | | | | b: 1 wt % | | | | | |
| | | | | HPC | | | | | |
| 22 | CaO | — | ethanol | b: 0.5 wt % | 20 | 1 | 7.6 | 0.6 | High |
| | | | | HPC | | | | | |
| 23 | MgO | — | ethanol | b: 0.5 wt % | 11 | 1 | 2.8 | 0.6 | High |
| | | | | HPC | | | | | |

^{a}Solid content is the result of parallel experiments instead of the calculated value before mixing.

### Example 4 Flow Reactor Test

The continuous flow reactor set-up used in the evaluation has been described in previous work, including WO 2017106916. It consists of a hydrogen reactor module, housing the catalytic inserts, a gas handling system a liquid delivery system (driven by a reagent pump, Gilson 305 HPLC pump), and electronic process control and data logging.

The reactor module contains twelve reactor zones, each of which are made from 15 cm long stainless steel tubing (Swagelok, 8 mm OD, 6 mm ID). It also contains five temperature probes (PT-100), situated along the length of eth reactor pathway.

The reactor module can be dismantled easily in order to facilitate change-over of the catalytic inserts. The reagent pump supplies the substrate feed stream, which contains a solution of the starting material substrate, neat or in a solvent. The hydrogen gas is supplied from a hydrogen cylinder (G-type cylinder) and mixed with the liquid stream in a T-piece. The pressure inside the reactor is regulated by a diaphragm back pressure regulator (BPR, Swagelok KBP1J0A4D5A20000), which is situated at the outlet of the reactor.

After passing through the BPR, the hot effluent can optionally be cooled in a coil type heat exchanger, which can operated with an appropriate cooling fluid. The product stream is then collected in a bottle or flask for further post processing and analysis.

Further safety components and process control and monitoring equipment is installed in the rig: safety pressure relief valve at reactor inlet (Swagelok, SS-4R3A); safety shut-down valve in the gas line (Burkert, 2/2-way solenoid valve 6027-A03); flash-back arrestor (Witt 85-10) in the gas line; mass flow controller in the gas line (Bronkhorst, MFC F-201CV-500); and pressure sensors in the liquid line, gas line and at the inlet of the reactor.

The reaction occurs at the solid-liquid interface of the catalytic inserts, inside the reactor zones. The operation of the reactor system is controlled by a dedicated LabView software. Temperature, pressure and gas flow rate are also monitored by the LabView control program.

In order to evaluate this reactor for hydrogenation reactions, a series of experiments were conducted investigating the hydrogenation of vinyl acetate (VAc), cinnamaldehyde (CAL), (−)-isopulegol (IPG), 1,4-butynediol (BYD) and 2-methyl-3-butyn-2-ol (MBY), using the solvents ethanol (EtOH), methanol (MeOH), isopropanol (iPrOH), n-heptane or ethyl acetate (EtOAc), see Scheme 1.

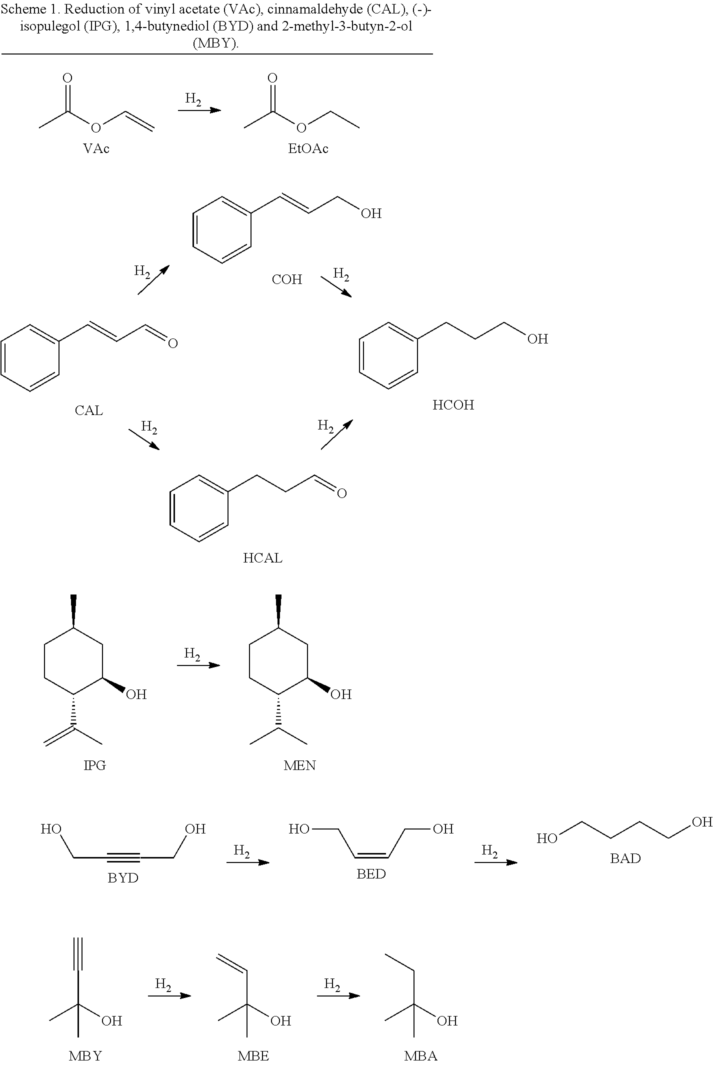

A typical hydrogenation reaction on the above reactor configuration was conducted as follows. First the 4 catalytic inserts inside the reactor were activated by flowing hydrogen over them at 24 bar, 120° C. and a gas flow rate of 190 mL_{N}/min. The activation the reactor was flushed with the solvent EtOH, using the liquid reagent pump. The substrate, VAc was dissolved in EtOH to a concentration of 2 mol/L.

Before start of the reaction, the hydrogen gas was introduced, together with the washing solvent EtOH, and the parameters for the reaction were adjusted: pressure inside reactor, p_{R}=20 bar, liquid flow rate, V_{L}=1 ml/min, gas flow rate V_{G}, =50 mL_{N}/min, and reactor temp, T_{R}=120° C. Once pressure and temperature had stabilised, the substrate (VAc) was fed into the reactor by changing over the reagent pump from pure solvent to the prepared clear stock solution. The clear product was collected at the outlet of the reactor in several fractions and was then analysed by ¹H-NMR and GC. Reaction conversions were calculated from ¹H NMR spectra, which were recorded on a Bruker AC-400 spectrometer in deuterated chloroform (from Cambridge Isotope Laboratories Inc.). The residual solvent peak at 6=7.26 ppm was used as an internal reference. Product compositions were analyzed by GC-FID and GC-MS.

The GC-FID results were also used to confirm NMR conversions and to calculate GC-based yields. GC-mass spectra were obtained with a Perkin Elmer Clarus 600 GC mass spectrometer using electron impact ionization in the positive ion mode with ionization energy of 70 eV. The gas chromatography was performed with a Perkin Elmer Elite-5MS GC column (30 m×0.25 mm ID, 0.25 μm film thickness), with a temperature program of 40° C. for 2 minutes, then heating at 10° C./min to 280° C. where the temperature was held for 4 minutes with a split ratio of 70, an injector temperature of 250° C. and the transfer line was set to 250° C. Ultra-high purity helium was used as the carrier gas with a flow rate of 0.7 ml/min.

GC-FID analysis was performed on an Agilent 6850 Series II gas chromatograph with a split/splitless inlet and a detector temperature of 250° C. Separation was done on a Grace BPX5 capillary column (25 m×0.32 mm ID, 0.50 μm film thickness), with a temperature program of 40° C. for 2 minutes, then heating at 10° C./min to 280° C. where the temperature was held for 4 minutes with a split ratio of 50 and an injector temperature of 200° C. High purity helium was used as the carrier gas with a flow rate of 2.4 ml/min. The reagents VAc, CAL, BYD and MBY were obtained from Sigma Aldrich; the solvents EtOH, MeOH, iPrOH, n-heptane and EtOAc were obtained from Merck KGaA. All reagents and solvents were used without further purification.

Table 5 shows experimental data from hydrogenation reactions of VAc and CAL and from hydrogenation reactions of (−)-isopulegol (IPG) to form (−)-menthol (MEN); Table 6 shows experimental data from the semi-hydrogenation reactions of BYD to BED; Table 7 shows experimental data from the semi-hydrogenation reactions of MBY to MBE. The experimental data advantageously shows that high conversion (VAc: up to 100%; CAL: up to 100%, BYD: up to 100%, MBY: up to 100%) and high selectivity (BYD: up to 92%, MBY: up to 100%) can be achieved using the coated catalytic static mixer scaffolds (CSMs) described herein. The space-time-yield of the catalytic static mixer reactor for these reactions was in the range of several kg/L h (VAc: up to 1.68 kg/L h; CAL: up to 0.70 kg/L h, BYD: up to 1.85 kg/L h, MBY: up to 3.22 kg/L h).

TABLE 5
Experimental data from the hydrogenation reactions using vinyl acetate (VAc)
and cinnamaldehyde (CAL) using either 4 or 12 CSMs

| | | | Pressure | Temp. | V_{L} | G/L | Conversion |
|---|---|---|---|---|---|---|---|
| | Catalyst | Substrate | [bar] | [° C.] | [ml/min] | [—] | [%] |
| 1 | 4× Pd/Al₂O₃ | VAc | 20 | 120 | 1 | 3.7 | 99 |
| 2 | 4× Pd/Al₂O₃ | VAc | 20 | 120 | 2 | 1.8 | 52 |
| 3 | 4× Pd/Al₂O₃ | CAL | 20 | 120 | 1 | 3.7 | 98 |
| 4 | 4× Pd/Al₂O₃ | CAL | 20 | 120 | 2 | 1.8 | 73 |
| 5 | 12× Pd/Al₂O₃ | VAc | 20 | 120 | 3 | 3.7 | 100 |
| 6 | 12× Pd/Al₂O₃ | VAc | 20 | 120 | 6 | 1.8 | 61 |
| 7 | 12× Pd/Al₂O₃ | CAL | 20 | 120 | 3 | 3.7 | 100 |
| 8 | 12× Pd/Al₂O₃ | CAL | 20 | 120 | 6 | 1.8 | 92 |
| 9 | 12× Pd/Al₂O₃ | IPG | 24 | 130 | 3 | 10.5 | 100 |
| 10 | 4× Pd-TiSiO₄ | VAc | 20 | 120 | 1 | 3.7 | 99 |
| 11 | 4× Pd-TiSiO₄ | VAc | 20 | 120 | 2 | 1.8 | 56 |
| 12 | 4× Pd-TiSiO₄ | CAL | 20 | 120 | 1 | 3.7 | 88 |
| 13 | 4× Pd-TiSiO₄ | CAL | 20 | 120 | 2 | 1.8 | 56 |

V_{L} = liquid volumetric flow rate;
G/L = volumetric ratio of gas to liquid at reactor pressure;
the concentration of substrate was 2 mol/L for VAc in EtOH and 1 mol/L for CAL in EtOAc and 4.5 mol/L for IPG in n-heptane.

TABLE 6
Experimental data from the hydrogenation reactions of 1,4-butynediol (BYD) to
selectively synthesise 1,4-butenediol (BED) using 4 CSMs

| | | | | | | | | (Z)- |
|---|---|---|---|---|---|---|---|---|
| | | p | T | V_{L} | H/S | | ene | alkene |
| Catalyst | Solvent | [bar] | [° C.] | [mL/min] | [mol/mol] | conversion | selectivity | selectivity |
| Pd-TiSiO₄ | 30% ⁱPrOH, | 17 | 55 | 1.0 | 7.20 | 100% | 31% | 65% |
| | 70% H₂O | | | | | | | |
| Pd-TiSiO₄ | 30% ⁱPrOH, | 24 | 100 | 0.5 | 27.18 | 100% | 2% | 72% |
| | 70% H₂O | | | | | | | |
| Pd-TiSiO₄ | 30% ⁱPrOH, | 24 | 100 | 0.5 | 14.40 | 100% | 6% | 67% |
| | 70% H₂O | | | | | | | |
| Pd-TiSiO₄ | 30% ⁱPrOH, | 17 | 55 | 1.5 | 4.80 | 100% | 49% | 78% |
| | 70% H₂O | | | | | | | |
| Pd-TiSiO₄ | 30% ⁱPrOH, | 12 | 55 | 1.5 | 4.80 | 97% | 71% | 90% |
| | 70% H₂O | | | | | | | |
| Pd-TiSiO₄ | 30% ⁱPrOH, | 12 | 55 | 1.5 | 1.83 | 87% | 80% | 93% |
| | 70% H₂O | | | | | | | |
| Pd-TiSiO₄ | 30% ⁱPrOH, | 12 | 55 | 1.5 | 3.00 | 91% | 79% | 93% |
| | 70% H₂O | | | | | | | |
| Pd-TiSiO₄ | 30% ⁱPrOH, | 8 | 55 | 1.5 | 1.83 | 65% | 83% | 94% |
| | 70% H₂O | | | | | | | |
| Pd-TiSiO₄ | 30% ⁱPrOH, | 8 | 55 | 1.0 | 2.75 | 87% | 78% | 92% |
| | 70% H₂O | | | | | | | |
| Pd-TiSiO₄ | 30% ⁱPrOH, | 8 | 55 | 0.5 | 5.49 | 100% | 49% | 74% |
| | 70% H₂O | | | | | | | |
| Pd-TiSiO₄ | 30% ⁱPrOH, | 12 | 55 | 2.5 | 3.89 | 81% | 81% | 93% |
| | 70% H₂O | | | | | | | |
| Pd-TiSiO₄ | 30% ⁱPrOH, | 12 | 55 | 2.5 | 3.00 | 70% | 84% | 94% |
| | 70% H₂O | | | | | | | |
| Pd-TiSiO₄ | 30% ⁱPrOH, | 10 | 55 | 2.0 | 2.50 | 70% | 83% | 94% |
| | 70% H₂O | | | | | | | |
| Pd-CaCO₃ | 30% ⁱPrOH, | 12 | 55 | 2.5 | 3.89 | 69% | 87% | 94% |
| Pb | 70% H₂O | | | | | | | |
| Pd-CaCO₃ | 30% ⁱPrOH, | 17 | 55 | 2.5 | 3.89 | 79% | 84% | 94% |
| Pb | 70% H₂O | | | | | | | |
| Pd-CaCO₃ | 30% ⁱPrOH, | 17 | 75 | 2.5 | 3.89 | 97% | 31% | 72% |
| Pb | 70% H₂O | | | | | | | |
| Pd-CaCO₃ | 30% ⁱPrOH, | 12 | 75 | 2.5 | 3.89 | 100% | 53% | 83% |
| Pb | 70% H₂O | | | | | | | |
| Pd-CaCO₃ | 30% ⁱPrOH, | 8 | 75 | 2.5 | 3.89 | 65% | 89% | 91% |
| Pb | 70% H₂O | | | | | | | |
| Pd-CaCO₃ | 30% ⁱPrOH, | 12 | 75 | 2.5 | 3.00 | 90% | 86% | 93% |
| Pb | 70% H₂O | | | | | | | |
| Pd-CaCO₃ | 30% ⁱPrOH, | 17 | 75 | 2.5 | 3.00 | 98% | 78% | 93% |
| Pb | 70% H₂O | | | | | | | |
| Pd-CaCO₃ | 30% ⁱPrOH, | 17 | 75 | 2.5 | 2.00 | 97% | 83% | 93% |
| Pb | 70% H₂O | | | | | | | |
| Pd-CaCO₃ | 30% ⁱPrOH, | 17 | 75 | 2.5 | 1.50 | 92% | 85% | 93% |
| Pb | 70% H₂O | | | | | | | |
| Pd-CaCO₃ | 30% ⁱPrOH, | 17 | 100 | 1.5 | 2.00 | 91% | 62% | 74% |
| Pb | 70% H₂O | | | | | | | |
| Pd-CaCO₃ | 30% ⁱPrOH, | 12 | 100 | 1.5 | 2.00 | 100% | 80% | 87% |
| Pb | 70% H₂O | | | | | | | |
| Pd-CaCO₃ | 30% ⁱPrOH, | 8 | 100 | 1.5 | 2.00 | 100% | 87% | 90% |
| Pb | 70% H₂O | | | | | | | |
| Pd-CaCO₃ | 30% ⁱPrOH, | 4 | 100 | 1.5 | 3.00 | 75% | 92% | 91% |
| Pb | 70% H₂O | | | | | | | |
| Pd-CaCO₃ | 30% ⁱPrOH, | 4 | 100 | 1.0 | 3.00 | 93% | 91% | 89% |
| Pb | 70% H₂O | | | | | | | |
| Pd-CaCO₃ | 30% ⁱPrOH, | 8 | 100 | 2.0 | 2.00 | 87% | 90% | 91% |
| Pb | 70% H₂O | | | | | | | |
| Pd-CaCO₃ | MeOH | 17 | 75 | 1.0 | 2.00 | 84% | 72% | 94% |
| Pb | | | | | | | | |
| Pd-CaCO₃ | MeOH | 20 | 100 | 1.5 | 2.00 | 76% | 81% | 91% |
| Pb | | | | | | | | |
| Pd-CaCO₃ | MeOH | 24 | 75 | 1.0 | 3.00 | 71% | 82% | 92% |
| Pb | | | | | | | | |
| Pd-CaCO₃ | MeOH | 24 | 110 | 0.7 | 3.00 | 100% | 64% | 79% |
| Pb | | | | | | | | |
| Pd-CaCO₃ | MeOH | 21 | 100 | 0.8 | 3.00 | 86% | 83% | 90% |
| Pb | | | | | | | | |
| Pd-CaCO₃ | MeOH | 24 | 90 | 0.9 | 3.00 | 82% | 87% | 92% |
| Pb | | | | | | | | |
| Pd-CaCO₃ | MeOH | 24 | 90 | 1.3 | 2.50 | 64% | 90% | 92% |
| Pb | | | | | | | | |
| Pd-CaCO₃ | MeOH | 24 | 100 | 1.2 | 2.50 | 74% | 88% | 93% |
| Pb | | | | | | | | |

p = reactor pressure;
T = reactor temperature;
V_{L} = liquid volumetric flow rate;
H/S = molar ratio of hydrogen to substrate;
the concentration of substrate was 0.31 mol/L for the iPrOH/H20 mixture and 6 mol/L for MeOH.

TABLE 7
Experimental data from the hydrogenation reactions of 2-methyl-3-butyn-2-ol
(MBY) to selectively synthesise 2-methyl-3-buten-2-ol (MBE) using CSMs

| | | concentration | p | T | V_{L} | H/S | | ene |
|---|---|---|---|---|---|---|---|---|
| Catalyst | Solvent | [M] | [bar] | [° C.] | [mL/min] | [mol/mol] | conversion | selectivity |
| Pd-TiSiO₄ | EtOH | 0.5 | 4 | 50 | 1.0 | 4.42 | 100% | 5% |
| Pd-TiSiO₄ | EtOH | 0.5 | 4 | 50 | 1.0 | 1.09 | 100% | 33% |
| Pd-TiSiO₄ | MeOH | 6.0 | 12 | 50 | 0.7 | 1.60 | 100% | 45% |
| Pd-TiSiO₄ | MeOH | 6.0 | 6 | 50 | 0.7 | 1.60 | 85% | 78% |
| Pd-TiSiO₄ | MeOH | 6.0 | 4 | 60 | 0.7 | 1.60 | 90% | 75% |
| Pd-TiSiO₄ | MeOH | 6.0 | 4 | 80 | 0.7 | 1.60 | 100% | 56% |
| Pd-TiSiO₄ | MeOH | 6.0 | 4 | 60 | 1.0 | 1.20 | 80% | 87% |
| Pd-TiSiO₄ | neat | 10.1 | 4 | 60 | 0.5 | 1.22 | 79% | 96% |
| Pd-TiSiO₄ | MeOH | 6.0 | 4 | 80 | 1.0 | 1.10 | 85% | 95% |
| Pd-TiSiO₄ | MeOH | 6.0 | 4 | 100 | 1.0 | 1.10 | 96% | 91% |
| Pd-TiSiO₄ | MeOH | 6.0 | 4 | 100 | 1.0 | 1.00 | 80% | 98% |
| Pd-TiSiO₄ | neat | 10.1 | 4 | 100 | 0.7 | 1.10 | 82% | 92% |
| Pd-TiSiO₄ | MeOH | 6.0 | 4 | 120 | 1.0 | 1.00 | 96% | 90% |
| Pd-TiSiO₄ | MeOH | 6.0 | 2 | 120 | 1.0 | 1.20 | 96% | 83% |
| Pd-TiSiO₄ | MeOH | 6.0 | 2 | 120 | 1.0 | 1.10 | 96% | 86% |
| Pd-TiSiO₄ | MeOH | 6.0 | 2 | 120 | 1.0 | 1.00 | 90% | 86% |
| Pd-CaCO₃ | MeOH | 6.0 | 2 | 40 | 0.50 | 1.78 | 23% | 100% |
| Pb | | | | | | | | |
| Pd-CaCO₃ | MeOH | 6.0 | 13 | 40 | 0.93 | 1.05 | 87% | 69% |
| Pb | | | | | | | | |
| Pd-CaCO₃ | MeOH | 6.0 | 2 | 40 | 1.31 | 1.00 | 10% | 100% |
| Pb | | | | | | | | |
| Pd-CaCO₃ | MeOH | 6.0 | 20 | 40 | 0.50 | 1.42 | 100% | 39% |
| Pb | | | | | | | | |
| Pd-CaCO₃ | MeOH | 6.0 | 16 | 49 | 0.50 | 2.55 | 100% | 7% |
| Pb | | | | | | | | |
| Pd-CaCO₃ | MeOH | 6.0 | 8 | 58 | 0.50 | 1.00 | 89% | 63% |
| Pb | | | | | | | | |
| Pd-CaCO₃ | MeOH | 6.0 | 7 | 74 | 0.75 | 1.73 | 100% | 53% |
| Pb | | | | | | | | |
| Pd-CaCO₃ | MeOH | 6.0 | 20 | 74 | 1.20 | 1.05 | 91% | 52% |
| Pb | | | | | | | | |
| Pd-CaCO₃ | MeOH | 6.0 | 2 | 81 | 0.90 | 1.00 | 36% | 94% |
| Pb | | | | | | | | |
| Pd-CaCO₃ | MeOH | 6.0 | 12 | 92 | 0.50 | 1.78 | 100% | 11% |
| Pb | | | | | | | | |
| Pd-CaCO₃ | MeOH | 6.0 | 19 | 109 | 0.62 | 1.00 | 71% | 46% |
| Pb | | | | | | | | |
| Pd-CaCO₃ | MeOH | 6.0 | 10 | 120 | 1.12 | 1.05 | 82% | 76% |
| Pb | | | | | | | | |
| Pd-CaCO₃ | MeOH | 6.0 | 20 | 120 | 0.67 | 1.85 | 100% | 3% |
| Pb | | | | | | | | |
| Pd-CaCO₃ | MeOH | 6.0 | 2 | 120 | 0.50 | 1.27 | 97% | 57% |
| Pb | | | | | | | | |
| Pd-CaCO₃ | MeOH | 6.0 | 2 | 120 | 0.50 | 2.55 | 97% | 11% |
| Pb | | | | | | | | |
| Pd-CaCO₃ | MeOH | 6.0 | 7 | 74 | 0.75 | 1.73 | 100% | 42% |
| Pb | | | | | | | | |
| Pd-CaCO₃ | neat | 10.1 | 10 | 120 | 1.00 | 1.27 | 74% | 56% |
| Pb | | | | | | | | |
| Pd-CaCO₃ | neat | 10.1 | 10 | 120 | 0.50 | 1.05 | 58% | 84% |
| Pb | | | | | | | | |
| Pd-CaCO₃ | neat | 10.1 | 15 | 120 | 0.50 | 1.05 | 89% | 70% |
| Pb | | | | | | | | |
| Pd-CaCO₃ | MeOH | 6.0 | 15 | 120 | 1.20 | 1.05 | 90% | 64% |
| Pb | | | | | | | | |
| Pd-CaCO₃ | MeOH | 6.0 | 15 | 100 | 1.00 | 1.05 | 83% | 61% |
| Pb | | | | | | | | |

p = reactor pressure;
T = reactor temperature;
V_{L} = liquid volumetric flow rate;
H/S = molar ratio of hydrogen to substrate.

### Example 5 Leaching Analysis

Collecting the product solution during the steady state regime of extended runs allowed catalyst leaching analysis of the catalytic static mixer scaffolds (CSMs).

A total of 1 L or more of the product solution was collected during the experiments for a standard test reaction, i.e. the reduction of VAc to EtOAc in ethanol. All organic materials were carefully removed and the remaining material was analysed by Inductively coupled plasma—optical emission spectrometry (ICP-EOS) for the presence of Cr, Mn, Fe, Mo, Al, Ni, Pd, Pt and Ru. The CSMs tested were all wash coated alumina type catalysts, (1) nickel on aluminium oxide, (2) palladium on aluminium oxide, (3) platinum on aluminium oxide, and (4) ruthenium on aluminium oxide, and 4 different sets of each of (1) to (4) CSMs were tested. Each CSM had a diameter of 6 mm and length of 150 mm. As shown in Table 7 below the product stream contains only parts-per-billion (ppb) levels of the active catalyst metals, Ni, Pd, Pt and Ru. It will be appreciated that the ppb values of Cr, Mn, Fe, Mo, and Al originate from the 316 stainless steel body of the reactor as well as from the base static mixer scaffold. It will also be appreciated that the ppb levels of the active metals, Ni, Pd, Pt and Ru, are from the catalytic coating deposited onto the CSMs.

The results unexpectedly and advantageously show that the active catalyst layer exhibits excellent adhesion to the base static mixer scaffold and that there is negligible degradation of the catalyst layer of the CSMs during continuous use over long periods of time. For all examples presented below in Table 8, the highest amount of soluble metal detected was Fe, hence the majority of the metal contamination is believed to come from the 316 stainless steel (SS) CSM scaffolds, the SS tubing and other SS parts of the reactor.

TABLE 8
Leaching data

| Catalyst | Cr | Mn | Fe | Mo | Al | Ni | Pd | Pt | Ru |
|---|---|---|---|---|---|---|---|---|---|
| (1) Ni/Al₂O₃ | 69 | 42 | 3728 | 16 | 988 | 1463 | BDL | BDL | BDL |
| (2) Pd/Al₂O₃ | 34 | 2 | 57 | 8 | 5 | 11 | 1 | N/A | N/A |
| (3) Pt/Al₂O₃ | 643 | 49 | 4888 | 112 | 3184 | 878 | BDL | 68 | BDL |
| (4) Ru/Al₂O₃ | 145 | 39 | 4566 | 20 | 322 | 217 | BDL | BDL | 316 |

Leaching data in ppb;
UD denotes below detectable level;
NA denotes not applicable.

1. A process for preparing a catalytically coated scaffold, comprising the steps of:
(i) applying a catalytic liquid suspension to a surface of a scaffold to provide a coating containing catalytically reactive sites on the surface of the coated scaffold, wherein the catalytic liquid suspension comprises a liquid carrier containing a plurality of ex-situ catalyst particles, and wherein the coated scaffold has a non-line-of-sight configuration comprising a plurality of passages configured for dispersing and mixing one or more fluidic reactants during flow and reaction thereof, and
(ii) drying the coated scaffold to remove the liquid carrier to provide a coated scaffold comprising ex-situ catalyst particles.

2. The process of claim 1, wherein the scaffold is a static mixer scaffold.

3. The process of claim 2, wherein the surface of the static mixer is pre-coated before step (i) with a support material and optionally a binder.

4. The process of any one of the preceding claims, wherein the catalytic liquid suspension further comprises a binder.

5. The process of any one of the preceding claims, wherein the step of applying the catalytic liquid suspension to the surface of the scaffold in step (i) is by wash-coating or dip-coating.

6. The process of any one of the preceding claims, wherein the process further comprises a pre-treatment step prior to applying the catalytic liquid suspension to the surface of the scaffold in step (i), and wherein the pre-treatment step comprises at least one surface treatment step to the surface of the scaffold selected from chemical treatment, anodic oxidation, hot dipping, vacuum plating, painting, thermal spraying, and acid etching.

7. The process of any one of the preceding claims, wherein the catalyst particles are formed from a catalyst material or a catalyst supported material comprising the catalyst material on a support material.

8. The process of claim 7, wherein the catalyst material is selected from a metal, metal oxide, aluminium silicate, activated carbon, mesoporous carbon, graphene, graphitic material, metal-organic framework, zeolite, or any combination thereof.

9. The process of claim 8, wherein the metal is selected from at least one of aluminium, iron, cerium, calcium, cobalt, copper, magnesium, zinc, nickel, palladium, platinum, gold, silicon, silver, ruthenium, iridium, rhodium, titanium, vanadium, zirconium, niobium, tantalum, and chromium, or a metal oxide thereof.

10. The process of any one of claims 3 to 9, wherein the support material is selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide.

11. The process of any one of claims 7 to 10, wherein the catalyst supported material is selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite.

12. The process of any one of the preceding claims, wherein the concentration of the catalyst particles in the catalytic liquid suspension is less than 10 wt. % based on the total weight of the catalytic liquid suspension.

13. The process of any one of claims 3 to 12, wherein the binder for the catalytic liquid suspension is selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide, or polyisobutylene.

14. The process of claim 13, wherein the binder is added at a concentration of between about 0.3 wt. % to about 5 wt. % based on the total weight of the catalytic liquid suspension.

15. The process of any one of the preceding claims, wherein the liquid carrier is selected from the group comprising water, ethanol, isopropanol, butanol, ethyl acetate, acetone or a combination thereof.

16. The process of any one of the preceding claims, wherein the catalytic liquid suspension has a solids content of between about 3 wt. % to about 28 wt. %.

17. The process of any one of the preceding claims, wherein the thickness of the coating is between about 1 μm to about 50 μm.

18. The process of any one of the preceding claims, wherein the surface area of the catalyst is between about 1 m²/g to about 1000 m²/g.

19. The process of any one of the preceding claims, wherein the adhesion of the coating provides a total mass loss of the coating of less than about 0.5 wt. % when measured by sonication testing.

20. The process of any one of the preceding claims, wherein the scaffold is a metal, metal alloy, cermet, carbon fibre, silicon carbide or polymer.

21. The process of claim 20, wherein the scaffold is a metal scaffold.

22. The process of claim 20, wherein the metal or metal alloy of the metal scaffold is titanium, aluminium or stainless steel.

23. The process of any one of the preceding claims, wherein the aspect ratio (L/d) of the scaffold is at least 75.

24. The process of any one of the preceding claims, wherein the ex-situ catalyst particles are less than about 5 μm.

25. The process of any one of the preceding claims, wherein the drying step comprises the steps of:
(a) applying a first temperature ranging between about 15° C. to about 30° C. to the coated surface of the scaffold for a first period of about 4 to 24 hours to volatilise at least a portion of volatile material from the catalytic liquid suspension; and
(b) applying a second temperature ranging between about 100° C. to about 180° C. under controlled atmosphere for a second period of about 4 to 24 hours such that a dried coating is formed on the surface of the scaffold.

26. A catalytically coated scaffold prepared by the process for preparing the catalytically coated scaffold of any one of claims 1 to 25.

27. A catalytically coated scaffold comprising a coating on a scaffold, wherein the coating comprises a plurality of catalyst particles, and wherein the coated scaffold has a non-line-of-sight configuration comprising a plurality of passages configured for dispersing and mixing one or more fluidic reactants during flow and reaction thereof.

28. The catalytically coated scaffold of claim 27, wherein the coating comprises a support material and optionally a binder.

29. The catalytically coated scaffold of claim 28, wherein the catalyst particles is selected from a metal, metal oxide, aluminium silicate, activated carbon, mesoporous carbon, graphene, graphitic material, metal-organic framework, zeolite, or any combination thereof.

30. The catalytically coated scaffold of claims 27 to 29, wherein the catalyst particles is a metal selected from at least one of aluminium, iron, cerium, calcium, cobalt, copper, magnesium, zinc, nickel, palladium, platinum, gold, silicon, silver, ruthenium, iridium, rhodium, titanium, vanadium, zirconium, niobium, tantalum, and chromium, or a metal oxide thereof.

31. The catalytically coated scaffold of claim 28, wherein the support material is selected from at least one of activated carbon, mesoporous carbon, graphene, graphitic material, metal-organic framework, zeolite, aluminium oxide, silicon dioxide, ceramic, magnesium chloride, calcium carbonate or dipotassium oxide.

32. The catalytically coated scaffold of claim 28, wherein the catalyst supported material is selected from at least one of ruthenium on aluminium oxide, palladium on aluminium oxide, lead-poisoned palladium on calcium carbonate, iron on aluminium oxide, silver on aluminium oxide, silica diphenyl phosphine palladium, palladium on titanium silicate, palladium on carbon, nickel modified aluminium oxide silicon oxide, or zeolite.

33. The catalytically coated scaffold of any one of claims 27 to 32, wherein the binder is selected from the group comprising hydroxypropyl cellulose, methyl cellulose, polyester, polyurethane, acrylic resins, condensation resins, polyvinyl acetate, poly(acrylic acid) sodium salt, polyvinylidene fluoride, polyethylene oxide, polyethylene glycol, dextrin, sodium silicate, colloidal silica, polydimethyl siloxane, boehmite, colloidal aluminium oxide, or polyisobutylene.

34. The catalytically coated scaffold of any one of claims 27 to 33, wherein the catalyst particles are less than 5 μm.

35. The catalytically coated scaffold of any one of claims 27 to 34, wherein the thickness of the coating is between about 1 μm to about 50 μm.

36. The catalytically coated scaffold of any one of claims 27 to 35, wherein the coated scaffold is a coated static mixer scaffold.

37. A continuous flow chemical reactor for use in reaction of one or more fluidic reactants, the reactor comprising one or more catalytically coated scaffolds prepared by the process of any one of claims 1 to 26 or the catalytically coated scaffold of any one of claims 27 to 36.

38. The continuous flow chemical reactor of claim 37, wherein the one or more fluidic reactants is provided as a continuous fluidic stream.

39. The continuous flow chemical reactor of claim 37 or 38, wherein the continuous fluidic stream is provided by at least one liquid phase.

40. A continuous flow process for a heterogeneous reaction comprising one or more chemical reactors as defined in any one of claims 37 to 39.

41. The continuous flow process of claim 40, wherein the heterogeneous reaction is a hydrogenation reaction.
